(19) United States
(12) Patent Application Publication
(10) Pub. No.: US 2017/0342021 A1
(43) Pub. Date: Nov. 30, 2017

(54) RAS-INHIBITING INDENYL ACETAMIDE COMPOUNDS, COMPOSITIONS, AND USES

(71) Applicant: ADT Pharmaceuticals, Inc., Orange Beach, AL (US)

(72) Inventors: Gary A. Piazza, Daphne, AL (US); Xi Chen, Hoover, AL (US); Adam B. Keeton, Gardendale, AL (US)

(21) Appl. No.: 15/537,222

(22) PCT Filed: Dec. 16, 2014
(86) PCT No.: PCT/US14/70511

§ 371 Date: Jun. 16, 2017

(51) Int. Cl. C07C 235/38; C07C 271/44; A61K 31/255; A61K 31/167; C07C 309/66; A61K 31/325; C07C 2602/08

(52) U.S. Cl. CPC C07C 235/38; C07C 309/66; C07C 271/44; A61K 31/325; A61K 31/255; A61K 31/167; C07C 2602/08

(57) ABSTRACT

Disclosed are compounds, for example, compounds of formula (I), wherein R, R₀ R₁-R₈, n, X, Y, Y¹, and E are as described herein, pharmaceutical compositions containing such compounds, and methods of treating or preventing a disease or condition, for example, cancer.

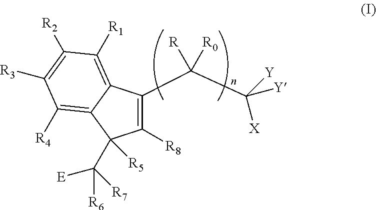

### STATEMENT REGARDING FEDERALLY SPONSORED RESEARCH AND DEVELOPMENT

This invention was made with partial support under NIH/NCI Grant Numbers CA 155638 and CA 148817. Therefore, the U.S. Government has certain rights in this invention.

### BACKGROUND OF THE INVENTION

Cancer is a leading cause of death in the developed world, with over one million people diagnosed and more than 500,000 deaths per year in the United States alone. Overall it is estimated that at least one in three people will develop some form of cancer during their lifetime. There are more than 200 different histopathological types of cancer, four of which (breast, lung, colorectal, and prostate) account for over half of all new cases in the U.S. (Jemal et al., Cancer J. Clin., 53, 5-26 (2003)).

Many of these tumors arise from mutations that activate Ras proteins, which control critically important cellular signaling pathways that regulate growth and other processes associated with tumorigenesis. The name “Ras” is an abbreviation of “Rat sarcoma” reflecting the way the first members of the Ras protein family were discovered. The name “ras” also is used to refer to the family of genes encoding these proteins.

Ras-driven cancers have remained the most intractable diseases to any available treatment. New therapeutic and preventative strategies are urgently needed for such cancers (Stephen et al., Cancer Cell, 25, 272-281 (2014)). Drug discovery programs worldwide have sought Ras-selective drugs for many years, but heretofore no avail (Spiegel et al., Nature Chem. Biol., 10, 613-622 (2014)). New drugs that selectively target abnormal or mutant Ras and/or Ras-mediated pathological processes in patients' tumors will enable highly efficacious treatments of such patients while minimizing toxicity to cells and tissues with normal Ras functions (Stephen et al., supra; Spiegel et al., supra).

Ras proteins are key regulators of several aspects of normal cell growth and malignant transformation, including cellular proliferation, survival and invasiveness, tumor angiogenesis and metastasis (Downward, Nature Rev. Cancer, 3, 11-22 (2003)). Ras proteins are abnormally active in most human tumors due to mutations in the ras genes themselves, or in upstream or downstream Ras pathway components, or other alterations in Ras signaling. Targeted therapies that inhibit Ras-mediated pathways therefore are expected to inhibit the growth, proliferation, survival and spread of tumor cells having activated or mutant Ras. Some such new experimental therapeutic agents have shown promising activity in preclinical studies, albeit with only modest activity in human clinical trials.

Genetic mutations in ras genes were first identified in human cancer over 3 decades ago. Such mutations result in the activation of one or more of three major Ras protein isoforms, including H-Ras, N-Ras, or K-Ras, that turn on signaling pathways leading to uncontrolled cell growth and tumor development. Activating ras gene mutations occur de novo in approximately one third of all human cancers and are especially prevalent in pancreatic, colorectal, and lung tumors. Ras mutations also develop in tumors that become resistant to chemotherapy and/or radiation, as well as to targeted therapies, such as receptor tyrosine kinase inhibitors (Gysin et al., Genes Cancer, 2, 359-372 (2011)). While ras mutations are relatively infrequent in other tumor types, for example, breast cancer, Ras can be pathologically activated by certain growth factor receptors that signal through Ras.

Although ras gene mutations have been known for many years, there currently are no available cancer therapeutics approved by the U.S. Food and Drug Administration that are known to selectively suppress the growth of tumors driven by activated Ras. In fact, Ras has been described as “undruggable” because of the relative abundance in cells and high affinity for its substrate, GTP (Takashima and Faller, Expert Opin. Ther. Targets, 17, 507-531 (2013)).

In addition to its role in cancer, activated Ras is important in a variety of other diseases, collectively referred to as “rasopathies.” One such disease, neurofibromatosis type 1 (NF1), a very prevalent autosomal dominant heritable disease, is caused by a mutation in neurofibromin, a Ras GAP (inactivating protein), which results in Ras hyperactivation in the relatively common event of loss of the second NF1 allele. Such mutations reportedly affect 1:3000 live births. The most dire symptoms associated with NF1 include numerous benign tumors (neurofibromas) arising from precursor nerve cells and Schwann cells of the peripheral nervous system. These tumors can cause severe problems depending on their location within the body, such as hearing or vision loss, as well as disfiguring masses on visible areas. Less common but extremely serious complications may arise when central nervous system gliomas develop or plexiform neurofibromas become transformed, resulting in the development of metastatic peripheral nerve sheath tumors (Tidyman and Rauen, Curr. Opin. Genet. Dev., 19, 230-236 (2009)). Another rare developmental disease which is attributable to hyperactive H-Ras is Costello syndrome. This condition causes a range of developmental abnormalities as well as predisposing patients to a variety of benign and malignant neoplasms (Tidyman and Rauen, supra).

Several approaches to treat diseases that arise from activating ras mutations have been undertaken. Because full maturation of the Ras protein requires lipid modification, attempts have been made to target this enzymatic process with inhibitors of farnesyl transferase and geranylgeranyltransferase, but with limited success and significant toxicity. Targeting of downstream components of Ras signaling with inhibitors of Raf/Mek/Erk kinase components of the cascading pathway has been an extremely active area of pharmaceutical research, but also fraught with difficulties and paradoxes arising from complex feedback systems within the pathways (Takashima and Faller, supra).

Inhibitors targeting components within the PI3K/Akt pathway also have not been successful as single agents, but presumably might synergize with Raf/Mek/Erk pathway inhibitors to block Ras-dependent tumor growth and survival. Similarly, several other molecular targets have been identified from RNAi screening, which might provide new opportunities to inhibit the growth of Ras-driven tumors; such other potential targets include CDK4, Cyclin D1, Tiam1, Myc, STK33, and TBK, as well as several genes involved in mitosis (Takashima and Faller, supra).

The nonsteroidal anti-inflammatory drug, sulindac (FIG. 1) has been reported to selectively inhibit proliferation of cultured tumor cells having ras mutations (Herrmann et al., Oncogene, 17, 1769-1776 (1998)). Extensive chemical modifications of sulindac and the related NSAID, indomethacin, have been aimed at removing cyclooxygenase-inhibitory activity, while improving anticancer activity (Gurpinar et al., Mol. Cancer Ther., 12, 663-674 (2013); Romeiro et al., Eur. J. Med. Chem., 44, 1959-1971 (2009); Chennamaneni et al., Eur. J. Med. Chem., 56, 17-29 (2012)). An example of a highly potent antiproliferative derivative is a hydroxy-substituted indene derivative of sulindac, OSIP-487703 (FIG. 1), that was reported to arrest colon cancer cells in mitosis by causing microtubule depolymerization (Xiao et al., Mol. Cancer Ther., 5, 60-67 (2006)). OSIP-487703 also was reported to inhibit the growth and induce apoptosis of human SW480 colon cancer cells. These properties of mitotic arrest and microtubule disruption were shared by several additional related compounds, including a pyridine (CP461) and trimethoxy (CP248) substituted variants (FIG. 1) (Lim et al., Clin. Cancer Res, 9, 4972-4982 (2003); Yoon, et al., Mol. Cancer Ther., 1, 393-404 (2002)). However, there was no reported association of antitumor properties of these compounds (FIG. 1) with Ras function, but rather such properties were attributed to direct binding to the microtubule subunit, tubulin, thereby causing mitotic arrest and blocking cell division. Still other reports describe their ability to induce apoptosis by inhibition of cGMP phosphodiesterase (Thompson et al., Cancer Research, 60, 3338-3342 (2000)).

Other investigators reported that sulindac sulfide (FIG. 1) can inhibit Ras-induced malignant transformation, possibly by decreasing the effects of activated Ras on its main effector, the c-Raf-lkinase, due to direct binding to the ras gene product p21 in a non-covalent manner (Herrmann et al., supra). Sulindac sulfide also can inhibit focus formation, a marker of malignant transformation, by rat or mouse fibroblasts by forced Ras expression, but not by other transformation pathways (Gala et al., Cancer Lett., 175, 89-94 (2002); Herrmann et al., supra). Sulindac sulfide was reported also to bind Ras directly and interfere with nucleotide exchange. Several groups additionally reported that sulindac interferes with Ras binding to the downstream signaling kinase c-Raf, and blocks activation of downstream signaling or transcription (Herrmann et al., supra; Pan et al., Cell Signal., 20, 1134-1141 (2008)).

The aforementioned findings led to efforts to improve the Ras inhibitory activity of sulindac sulfide through chemical modifications (Karaguni et al., Bioorg. Med. Chem. Lett., 12, 709-713 (2002)). Several derivatives were identified that were more potent inhibitors of tumor cell proliferation, and four related compounds (FIG. 2) exhibited selectivity towards a Ras-transfected MDCK cell line compared to the parental cell line. Three of these compounds also potently disrupted the Ras-Raf interaction. However, none of the four were more potent toward the mutant K-Ras-bearing SW-480 cell line, although they did inhibit Erk phosphorylation and bound weakly to the G-domain of H-Ras (Waldmann et al., Angew. Chem. Int. Ed. Engl., 43, 454-458 (2004)).

In addition to sulindac sulfide, the non-COX inhibitory sulfone metabolite of sulindac has been reported to have selective effects on tumor cells with mutant Ras. For example, transfection of Caco-2 colon tumor cells with the activated K-Ras oncogene caused cells treated with either sulindac sulfide or sulfone to undergo apoptosis earlier than non-transfected cell (Lawson et al., Cancer Epidemiol. Biomarkers Prev., 9, 1155-62 (2000)). Other investigators have reported that sulindac sulfone can inhibit mammary tumorigenesis in rats and that the effect was greater on tumors with the mutant H-Ras genotype (Thompson et al., Cancer Research 57, 267-271 (1997)). However, other investigators report that the inhibition of colon tumorigenesis in rats by either sulindac or sulindac sulfone occurs independently of K-Ras mutations (de Jong et al., Amer. J. Physio. Gastro and Liver Phys. 278, 266-272 (2000)). Yet other investigators report that the K-Ras oncogene increases resistance to sulindac-induced apoptosis in rat enterocytes (Arber et al., Gastroenterology, 113, 1892-1900 (1997)). As such, the influence of Ras mutations on the anticancer activity of sulindac and its metabolites is controversial and unresolved, and has not been exploited to improve anticancer potency or selectivity.

Certain other compounds have been described with selective toxicity toward cells expressing activated Ras. A high-throughput phenotypic screen of over 300,000 compounds was conducted within NIH Molecular Libraries Screening Center program to identify compounds which were synthetically lethal to cells expressing oncogenic H-Ras. A lead compound, ML210 (FIG. 3), inhibited growth of cells expressing mutant Ras with an IC50 of 71 nM, and was 4-fold selective versus cells lacking oncogenic Ras. Though the specific molecular target of ML210 is unknown, the compound was chemically optimized to eliminate reactive groups and improve pharmacologic properties (ML210, 12/12/2011 update, Probe Reports from NIH Molecular Libraries Program, Bethesda, http://www.ncbi.nlm.nih.gov/books/NBK98919/).

A separate high-throughput screen identified two compounds, RSL3 and RSL5 (FIG. 3) which induce non-apoptotic, Mek-dependent, oxidative cell death (Yang and Stockwell, Chem. Biol., 15, 234-245 (2008). RSL5, like a previously identified Ras synthetic lethal compound, erastin (FIG. 3), binds the voltage-dependent anion channel (VDAC) (Dolma et al., Cancer Cell, 3, 285-296 (2003)). Yet another small-molecule screen identified oncrasin, a compound selectively active against K-Ras mutant cell lines (Guo et al., Cancer Res., 68, 7403-7408 (2008)). One analog, NSC-743380 (FIG. 3), is highly potent and has shown antitumor activity in a preclinical model of K-Ras driven renal cancer (Guo et al., PLoS One, 6, e28487 (2011)). A prodrug approach has recently been described for oncrasin derivatives, to improve stability, pharmacokinetics, and safety (Wu et al., Bioorg. Med. Chem., 22, 5234-5240 (2014)). A synthetic lethal screen using embryonic fibroblasts derived from mice expressing the oncogenic K-Ras (G12D) identified a compound, lanperisone (FIG. 3), that induced non-apoptotic cell death via a mechanism involving oxidative stress (Shaw et al., Proc. Natl. Acad. Sci. USA, 108, 8773-8778 (2011)). In contrast to the synthetic lethal approach, a fragment-based screening approach paired with crystallographic studies has been used to identify compounds which irreversibly bind to and inhibit K-Ras in lung tumor cells having the relatively rare G12C ras gene mutation (Ostrem et al., Nature, 503, 548-551 (2013)). While compounds of this series potently inhibit Ras through a covalent interaction, the low frequency of this mutation may limit the utility of such compounds. Finally, a new investigational strategy for targeting oncogenic Ras has been described (Zimmerman et al., J. Med. Chem., 57, 5435-5448 (2014)) which involves structure guided design and kinetic analysis of benzimidazole inhibitors targeting the PDEδ prenyl binding site.

WO 97/47303; WO 2014/047592 and U.S. Patent Application Publication Nos. 2003/0009033 and 2003/0194750, and U.S. Pat. Nos. 6,063,818; 6,071,934, 5,965,619; 5,401,774; 6,538,029; and 6,121,321 and UK Patent No. GB 1370028 disclose certain anticancer compounds; however, these documents do not disclose that the compounds have any Ras-specific activity, nor any basis for a selective Ras-directed method of use.

The foregoing shows that there exists an unmet need for compounds that are suitable for treating or preventing cancers. There further exists an unmet need for compounds that inhibit Ras-dependent diseases or undesirable conditions.

### BRIEF SUMMARY OF THE INVENTION

The invention provides a compound of formula I:

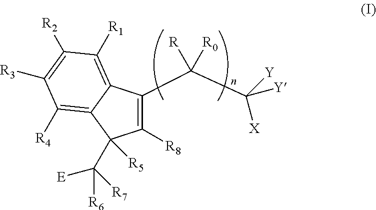

wherein:

R and R₀ are, independently, hydrogen or hydroxyl; n is 0, 1 or 2;

three of R₁, R₂, R₃, and R₄ are hydrogens, and one is halogen, alkyl, or alkoxy, or two of R₁, R₂, R₃, and R₄ are hydrogens and two are alkoxy;

R₅ and R₆ together form a carbon-carbon bond; R₇ is hydrogen; R₈ is alkyl;

Y and Y′ together is oxygen;

X is NR′R″, where R′ is aryl substituted with one or more of halogen, alkyl, haloalkyl, cyano, cyanoalkyl, nitro, hydroxyl, alkoxy, formyloxy, hydroxyalkyl, aldehydo, amino, alkylamino, dialkylamino, aminoalkyl, alkylaminoalkyl, mercapto, and alkylmercapto, azido, and substituted or unsubstituted groups selected from alkylsulfonyl, alkylsulfinyl, alkylsulfinyloxy, alkylsulfonyloxy, alkylcarbonyloxy, carbamate, carbamido, alkoxycarbonyl, alkylaminocarbonyl, aminocarbonyl, and sulfonamido, or any two substituted positions may be occupied by an alkylenedioxy group; and R″ is hydrogen; and

E is a substituted aryl; or

a pharmaceutically acceptable salt thereof or a prodrug thereof, or a corresponding Z or E-isomer.

The invention further provides a compound of the formula (II):

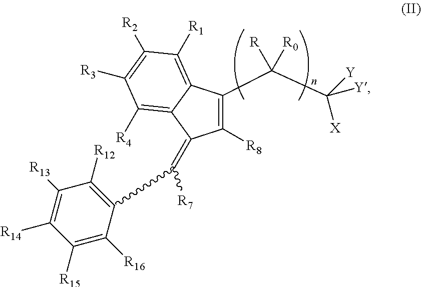

wherein:

R and R₀ are, independently, hydrogen or hydroxyl; n is 0, 1 or 2;

Y and Y′ together is oxygen;

three of R₁, R₂, R₃, and R₄ are hydrogens, and one is halogen, alkyl, or alkoxy, or two of R₁, R₂, R₃, and R₄ are hydrogens and two are alkoxy;

R₇ is hydrogen and R₈ is alkyl;

at least one of R₁₂, R₁₃, R₁₄, R₁₅, and R₁₆ is independently selected from halogen, alkyl, haloalkyl, cyano, cyanoalkyl, nitro, hydroxyl, alkoxy, formyloxy, hydroxyalkyl, aldehydo, amino, alkylamino, aminoalkyl, alkylaminoalkyl, dialkylamino, mercapto, alkylmercapto, azido, and substituted or unsubstituted groups selected from alkylsulfonyl, alkylsulfinyl, alkylsulfinyloxy, alkylsulfonyloxy, alkylcarbonyloxy, carbamate, carbamido, alkoxycarbonyl, alkylaminocarbonyl, aminocarbonyl, and sulfonamido, or any two of R₁₂, R₁₃, R₁₄, R₁₅ and R₁₆ form an alkylenedioxy group; and

X is NR′R″, where R″ is hydrogen; R′ is aryl substituted with one or more of halogen, alkyl, haloalkyl, cyano, cyanoalkyl, nitro, hydroxyl, alkoxy, formyloxy, hydroxyalkyl, aldehydo, amino, alkylamino, dialkylamino, aminoalkyl, alkylaminoalkyl, mercapto, and alkylmercapto, azido, and substituted or unsubstituted groups selected from alkylsulfonyl, alkylsulfinyl, alkylsulfinyloxy, alkylsulfonyloxy, alkylcarbonyloxy, carbamate, carbamido, alkoxycarbonyl, alkylaminocarbonyl, aminocarbonyl, and sulfonamido, or any two substituted positions may be occupied by an alkylenedioxy group;

or a pharmaceutically acceptable salt, or a prodrug thereof, or a corresponding Z or E-isomer.

The invention also provides a method of inhibiting a human or nonhuman mammalian Ras-mediated biological process, which method comprising administering in vivo or in vitro a Ras-inhibitory amount of at least one compound of formula I, a pharmaceutically acceptable salt or prodrug thereof:

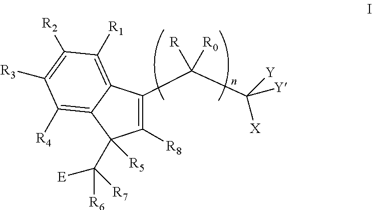

wherein:

R and R₀ are, independently, hydrogen or hydroxyl; n is 0, 1 or 2;

three of R₁, R₂, R₃, and R₄ are hydrogens, and one is halogen, alkyl, or alkoxy, or two of R₁, R₂, R₃, and R₄ are hydrogens and two are alkoxy;

R₅ and R₆ together form a carbon-carbon bond; R₇ is hydrogen; R₈ is alkyl;

Y and Y′ together is oxygen;

X is NR′R″, where R′ is aryl substituted with one or more of halogen, alkyl, haloalkyl, cyano, cyanoalkyl, nitro, hydroxyl, alkoxy, formyloxy, hydroxyalkyl, aldehydo, amino, alkylamino, dialkylamino, aminoalkyl, alkylaminoalkyl, mercapto, and alkylmercapto, azido, and substituted or unsubstituted groups selected from alkylsulfonyl, alkylsulfinyl, alkylsulfinyloxy, alkylsulfonyloxy, alkylcarbonyloxy, carbamate, carbamido, alkoxycarbonyl, alkylaminocarbonyl, aminocarbonyl, and sulfonamido, or any two substituted positions may be occupied by an alkylenedioxy group; and R″ is hydrogen; and

E is a substituted aryl;

wherein the compound of formula I or pharmaceutically acceptable salt or prodrug thereof or Z or E-isomer is administered alone or in combination with at least one additional therapeutic agent other than said compound of formula I or pharmaceutically acceptable salt or prodrug thereof or Z or E-isomer.

The present invention further provides a method of therapeutically or prophylactically treating a human or nonhuman mammalian patient with a disease or condition treatable by inhibition of one or more Ras-mediated biological process, which method comprising administering to a patient in need thereof a therapeutically or prophylactically effective amount of at least one ras-inhibitory compound of formula I, a pharmaceutically acceptable salt or prodrug thereof:

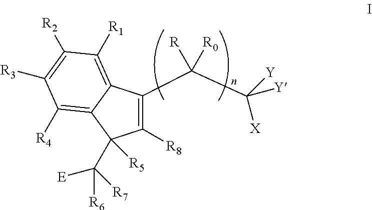

wherein:

R and R₀ are, independently, hydrogen or hydroxyl; n is 0, 1 or 2;

three of R₁, R₂, R₃, and R₄ are hydrogens, and one is halogen, alkyl, or alkoxy, or two of R₁, R₂, R₃, and R₄ are hydrogens and two are alkoxy;

R₅ and R₆ together form a carbon-carbon bond; R₇ is hydrogen; R₈ is alkyl;

Y and Y′ together is oxygen;

X is NR′R″, where R′ is aryl substituted with one or more of halogen, alkyl, haloalkyl, cyano, cyanoalkyl, nitro, hydroxyl, alkoxy, formyloxy, hydroxyalkyl, aldehydo, amino, alkylamino, dialkylamino, aminoalkyl, alkylaminoalkyl, mercapto, and alkylmercapto, azido, and substituted or unsubstituted groups selected from alkylsulfonyl, alkylsulfinyl, alkylsulfinyloxy, alkylsulfonyloxy, alkylcarbonyloxy, carbamate, carbamido, alkoxycarbonyl, alkylaminocarbonyl, aminocarbonyl, and sulfonamido, or any two substituted positions may be occupied by an alkylenedioxy group; and R″ is hydrogen; and

E is a substituted aryl;

wherein the compound of formula I or pharmaceutically acceptable salt or prodrug thereof or Z or E-isomer is administered alone or in combination with at least one additional therapeutic agent other than said compound of formula I or pharmaceutically acceptable salt or prodrug thereof or Z or E-isomer.

A method of inhibiting a human or nonhuman mammalian Ras-mediated biological process, which method comprising administering in vivo or in vitro a Ras-inhibitory amount of at least one compound selected from:
(Z)-2-(1-(4-hydroxy-3,5-dimethoxybenzylidene)-5-methoxy-2-methyl-1H-inden-3-yl)-N-(m-tolyl)acetamide (036),
(Z)-2-(5-fluoro-1-(4-hydroxy-3,5-dimethoxybenzylidene)-2-methyl-1H-inden-3-yl)-N-(m-tolyl)acetamide (037),
(Z)—N-(3,4-dimethylphenyl)-2-(1-(4-hydroxy-3,5-dimethoxybenzylidene)-5-methoxy-2-methyl-1H-inden-3-yl)acetamide (038),
(Z)—N-(3,4-dimethylphenyl)-2-(5-fluoro-1-(4-hydroxy-3,5-dimethoxybenzylidene)-2-methyl-1H-inden-3-yl)acetamide (039),
(Z)-2-(1-(4-hydroxy-3,5-dimethoxybenzylidene)-5-methoxy-2-methyl-1H-inden-3-yl)-N-(p-tolyl)acetamide (040),
(Z)-2-(5-fluoro-1-(4-hydroxy-3,5-dimethoxybenzylidene)-2-methyl-1H-inden-3-yl)-N-(p-tolyl)acetamide (041),
(Z)-2-(1-(4-hydroxy-3,5-dimethoxybenzylidene)-5-methoxy-2-methyl-1H-inden-3-yl)-N-(o-tolyl)acetamide (042),
(Z)-2-(5-fluoro-1-(4-hydroxy-3,5-dimethoxybenzylidene)-2-methyl-1H-inden-3-yl)-N-(o-tolyl)acetamide (043),
(Z)-2-(1-(4-hydroxy-3,5-dimethoxybenzylidene)-5-methoxy-2-methyl-1H-inden-3-yl)-N-(3-methoxyphenyl)acetamide (044),
(Z)-2-(5-fluoro-1-(4-hydroxy-3,5-dimethoxybenzylidene)-2-methyl-1H-inden-3-yl)-N-(3-methoxyphenyl)acetamide (045),
(Z)-2-(1-(4-hydroxy-3-methoxybenzylidene)-5-methoxy-2-methyl-1H-inden-3-yl)-N-(m-tolyl)acetamide (046),
(Z)-2-(5-fluoro-1-(4-hydroxy-3-methoxybenzylidene)-2-methyl-1H-inden-3-yl)-N-(m-tolyl)acetamide (047),
(Z)-2-(1-(4-hydroxy-3-methoxybenzylidene)-5-methoxy-2-methyl-1H-inden-3-yl)-N-(3-methoxyphenyl)acetamide (048),
(Z)-2-(5-fluoro-1-(4-hydroxy-3-methoxybenzylidene)-2-methyl-1H-inden-3-yl)-N-(3-methoxyphenyl)acetamide (049),
(Z)-2-(5-fluoro-1-(4-hydroxy-3,5-dimethoxybenzylidene)-2-methyl-1H-inden-3-yl)-N-(7-methylnaphthalen-2-yl)acetamide (050),
(Z)-4-((5-fluoro-2-methyl-3-(2-oxo-2-(m-tolylamino)ethyl)-1H-inden-1-ylidene)methyl)-2,6-dimethoxyphenyl ethylcarbamate (051),
(Z)-4-((5-fluoro-2-methyl-3-(2-oxo-2-(m-tolylamino)ethyl)-1H-inden-1-ylidene)methyl)-2,6-dimethoxyphenyl methanesulfonate (052),
(Z)-2-(5-fluoro-1-(4-acetoxy-3,5-dimethoxybenzylidene)-2-methyl-1H-inden-3-yl)-N-(m-tolyl)acetamide (053),
(Z)-2-(5-fluoro-1-(4-hydroxy-3,5-dimethoxybenzylidene)-2-methyl-1H-inden-3-yl)-N-(4-fluorophenyl)acetamide (054),
(Z)—N-(4-chlorophenyl)-2-(5-fluoro-1-(4-hydroxy-3,5-dimethoxybenzylidene)-2-methyl-1H-inden-3-yl)acetamide (055),
(Z)—N-(3,4-dichlorophenyl)-2-(5-fluoro-1-(4-hydroxy-3,5-dimethoxybenzylidene)-2-methyl-1H-inden-3-yl)acetamide (056),
(Z)—N-(4-bromophenyl)-2-(5-fluoro-1-(4-hydroxy-3,5-dimethoxybenzylidene)-2-methyl-1H-inden-3-yl)acetamide (057),
(Z)—N-(3-bromophenyl)-2-(5-fluoro-1-(4-hydroxy-3,5-dimethoxybenzylidene)-2-methyl-1H-inden-3-yl)acetamide (058), and
(Z)—N-(4-(tert-butyl)phenyl)-2-(5-fluoro-1-(4-hydroxy-3,5-dimethoxybenzylidene)-2-methyl-1H-inden-3-yl)acetamide (059), or

a pharmaceutically acceptable salt or prodrug thereof, or the corresponding E-isomer thereof,

wherein said compound, pharmaceutically acceptable salt or prodrug thereof or E-isomer thereof, is administered alone or in combination with at least one additional therapeutic agent other than said compound, pharmaceutically acceptable salt, prodrug, or E-isomer thereof.

The present invention further provides a method of therapeutically or prophylactically treating a human or nonhuman mammalian patient with a disease or condition treatable by inhibition of one or more Ras-mediated biological process, which method comprising administering to a patient in need thereof a therapeutically or prophylactically effective amount of at least one ras-inhibitory compound selected from:
(Z)-2-(1-(4-hydroxy-3,5-dimethoxybenzylidene)-5-methoxy-2-methyl-1H-inden-3-yl)-N-(m-tolyl)acetamide (036),
(Z)-2-(5-fluoro-1-(4-hydroxy-3,5-dimethoxybenzylidene)-2-methyl-1H-inden-3-yl)-N-(m-tolyl)acetamide (037),
(Z)—N-(3,4-dimethylphenyl)-2-(1-(4-hydroxy-3,5-dimethoxybenzylidene)-5-methoxy-2-methyl-1H-inden-3-yl)acetamide (038),
(Z)—N-(3,4-dimethylphenyl)-2-(5-fluoro-1-(4-hydroxy-3,5-dimethoxybenzylidene)-2-methyl-1H-inden-3-yl)acetamide (039),
(Z)-2-(1-(4-hydroxy-3,5-dimethoxybenzylidene)-5-methoxy-2-methyl-1H-inden-3-yl)-N-(p-tolyl)acetamide (040),
(Z)-2-(5-fluoro-1-(4-hydroxy-3,5-dimethoxybenzylidene)-2-methyl-1H-inden-3-yl)-N-(p-tolyl)acetamide (041),
(Z)-2-(1-(4-hydroxy-3,5-dimethoxybenzylidene)-5-methoxy-2-methyl-1H-inden-3-yl)-N-(o-tolyl)acetamide (042),
(Z)-2-(5-fluoro-1-(4-hydroxy-3,5-dimethoxybenzylidene)-2-methyl-1H-inden-3-yl)-N-(o-tolyl)acetamide (043),
(Z)-2-(1-(4-hydroxy-3,5-dimethoxybenzylidene)-5-methoxy-2-methyl-1H-inden-3-yl)-N-(3-methoxyphenyl)acetamide (044),
(Z)-2-(5-fluoro-1-(4-hydroxy-3,5-dimethoxybenzylidene)-2-methyl-1H-inden-3-yl)-N-(3-methoxyphenyl)acetamide (045),
(Z)-2-(1-(4-hydroxy-3-methoxybenzylidene)-5-methoxy-2-methyl-1H-inden-3-yl)-N-(m-tolyl)acetamide (046),
(Z)-2-(5-fluoro-1-(4-hydroxy-3-methoxybenzylidene)-2-methyl-1H-inden-3-yl)-N-(m-tolyl)acetamide (047),
(Z)-2-(1-(4-hydroxy-3-methoxybenzylidene)-5-methoxy-2-methyl-1H-inden-3-yl)-N-(3-methoxyphenyl)acetamide (048),
(Z)-2-(5-fluoro-1-(4-hydroxy-3-methoxybenzylidene)-2-methyl-1H-inden-3-yl)-N-(3-methoxyphenyl)acetamide (049),
(Z)-2-(5-fluoro-1-(4-hydroxy-3,5-dimethoxybenzylidene)-2-methyl-1H-inden-3-yl)-N-(7-methylnaphthalen-2-yl)acetamide (050),
(Z)-4-((5-fluoro-2-methyl-3-(2-oxo-2-(m-tolylamino)ethyl)-1H-inden-1-ylidene)methyl)-2,6-dimethoxyphenyl ethylcarbamate (051),
(Z)-4-((5-fluoro-2-methyl-3-(2-oxo-2-(m-tolylamino)ethyl)-1H-inden-1-ylidene)methyl)-2,6-dimethoxyphenyl methanesulfonate (052),
(Z)-2-(5-fluoro-1-(4-acetoxy-3,5-dimethoxybenzylidene)-2-methyl-1H-inden-3-yl)-N-(m-tolyl)acetamide (053),
(Z)-2-(5-fluoro-1-(4-hydroxy-3,5-dimethoxybenzylidene)-2-methyl-1H-inden-3-yl)-N-(4-fluorophenyl)acetamide (054),
(Z)—N-(4-chlorophenyl)-2-(5-fluoro-1-(4-hydroxy-3,5-dimethoxybenzylidene)-2-methyl-1H-inden-3-yl)acetamide (055),
(Z)—N-(3,4-dichlorophenyl)-2-(5-fluoro-1-(4-hydroxy-3,5-dimethoxybenzylidene)-2-methyl-1H-inden-3-yl)acetamide (056),
(Z)—N-(4-bromophenyl)-2-(5-fluoro-1-(4-hydroxy-3,5-dimethoxybenzylidene)-2-methyl-1H-inden-3-yl)acetamide (057),
(Z)—N-(3-bromophenyl)-2-(5-fluoro-1-(4-hydroxy-3,5-dimethoxybenzylidene)-2-methyl-1H-inden-3-yl)acetamide (058), and
(Z)—N-(4-(tert-butyl)phenyl)-2-(5-fluoro-1-(4-hydroxy-3,5-dimethoxybenzylidene)-2-methyl-1H-inden-3-yl)acetamide (059), or

a pharmaceutically acceptable salt or prodrug thereof, or the corresponding E-isomer thereof,

wherein said compound, pharmaceutically acceptable salt or prodrug thereof, or E-isomer thereof, is administered alone or in combination with at least one additional therapeutic agent other than said compound, pharmaceutically acceptable salt, prodrug, or E-isomer thereof.

The compounds of the invention are suitable for treating or preventing cancer.

The present invention further provides a pharmaceutical composition comprising a compound described above and a pharmaceutically acceptable carrier.

### BRIEF DESCRIPTION OF THE SEVERAL VIEWS OF THE DRAWING(S)

FIG. 1 depicts the chemical structures of sulindac and certain derivatives thereof reportedly having anticancer activity.

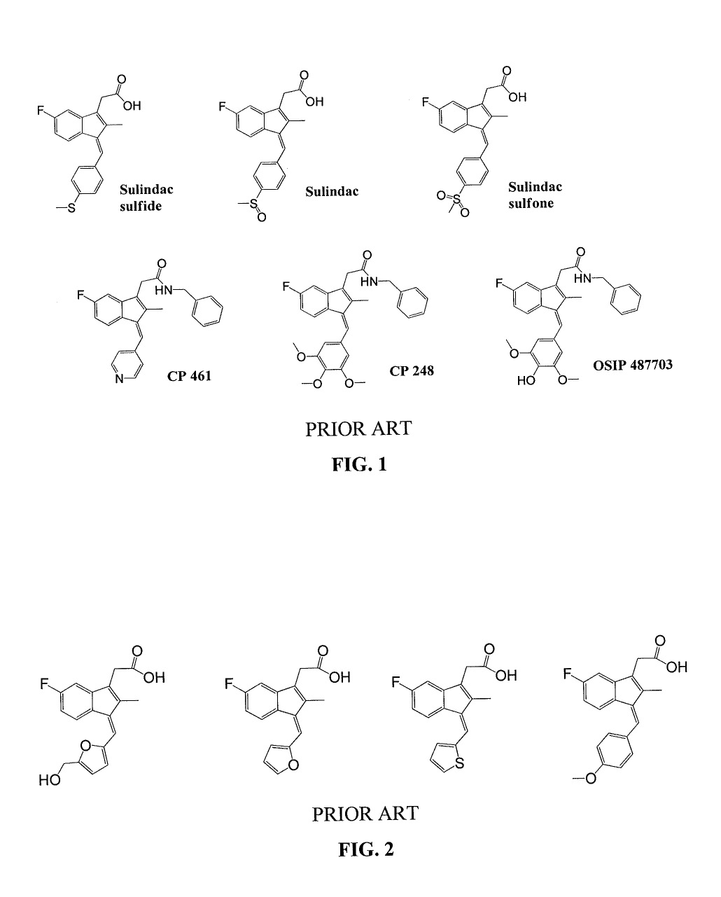

FIG. 2 depicts the chemical structures of certain other sulindac derivatives reported to inhibit Ras.

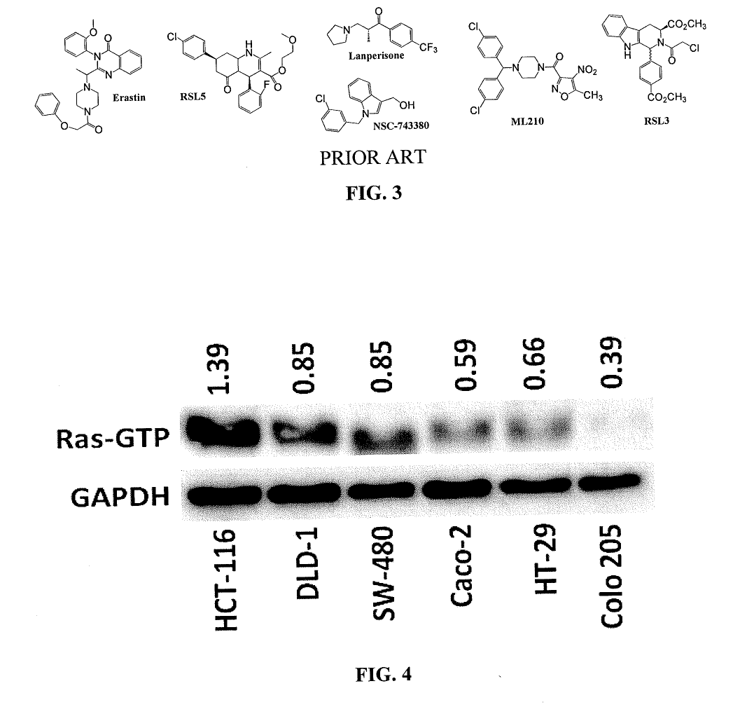

FIG. 3 shows chemical structures of selective Ras-inhibitory compounds identified by synthetic lethal screening.

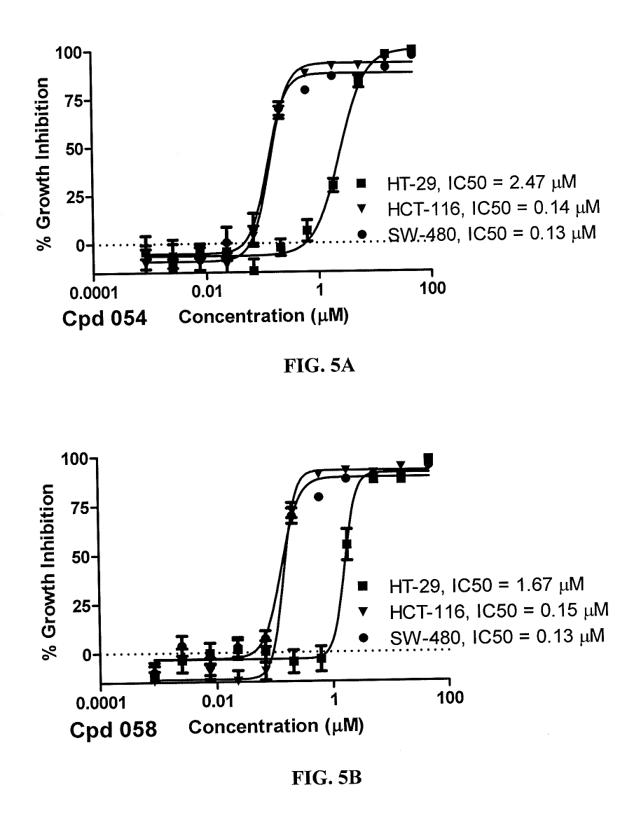

FIG. 4 depicts the results of a Ras Binding Domain (RBD) pulldown assay paired with Western blot showing the relative levels of Ras activation in a panel of colorectal cancer cell lines.

FIGS. 5A-5E show Ras-selective tumor cell growth-inhibiting activity of exemplary Ras-inhibitory compounds 054 (FIG. 5A), 058 (FIG. 5B), 057 (FIG. 5C), 055 (FIG. 5D), and 056 (FIG. 5E) against human HCT-116 and SW-480 colon tumor cells expressing mutant Ras, compared to human HT-29 colon tumor cells expressing wild-type Ras.

### DETAILED DESCRIPTION OF THE INVENTION

In accordance with an embodiment, the invention provides a compound of formula I:

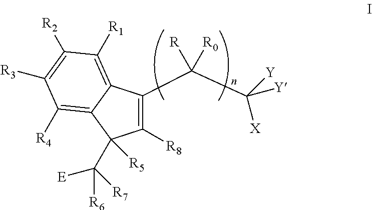

wherein:

R and R₀ are, independently, hydrogen or hydroxyl; n is 0, 1 or 2;

three of R₁, R₂, R₃, and R₄ are hydrogens, and one is halogen, alkyl, or alkoxy, or two of R₁, R₂, R₃, and R₄ are hydrogens and two are alkoxy;

R₅ and R₆ together form a carbon-carbon bond; R₇ is hydrogen; R₈ is alkyl;

Y and Y′ together is oxygen;

X is NR′R″, where R′ is aryl substituted with one or more of halogen, alkyl, haloalkyl, cyano, cyanoalkyl, nitro, hydroxyl, alkoxy, formyloxy, hydroxyalkyl, aldehydo, amino, alkylamino, dialkylamino, aminoalkyl, alkylaminoalkyl, mercapto, and alkylmercapto, azido, and substituted or unsubstituted groups selected from alkylsulfonyl, alkylsulfinyl, alkylsulfinyloxy, alkylsulfonyloxy, alkylcarbonyloxy, carbamate, carbamido, alkoxycarbonyl, alkylaminocarbonyl, aminocarbonyl, and sulfonamido, or any two substituted positions may be occupied by an alkylenedioxy group; and R″ is hydrogen; and

E is a substituted aryl; or

a pharmaceutically acceptable salt thereof or a prodrug thereof, or a corresponding Z or E-isomer.

In one aspect, when R and R₀ are hydrogen, n is 1, three of R₁, R₂, R₃, and R₄ are hydrogens and one is halogen, alkyl, or alkoxy or two of R₁, R₂, R₃ and R₄ are hydrogens and two are alkoxy, R₅ and R₆ together form a carbon-carbon bond, R₇ is hydrogen, R₈ is alkyl, Y and Y′ together is oxygen, X is NR′R″ wherein R″ is hydrogen, and R′ is a substituted aryl, then E cannot be a substituted aryl. For example, when R and R₀ are hydrogen, n is 1, three of R₁, R₂, R₃, and R₄ are hydrogens and one is halogen, alkyl, or alkoxy or two of R₁, R₂, R₃ and R₄ are hydrogens and two are alkoxy, R₅ and R₆ together form a carbon-carbon bond, R₇ is hydrogen, R₈ is alkyl, Y and Y′ together is oxygen, X is NR′R″ wherein R″ is hydrogen, R′ is an aryl substituted with any of halo, alkoxy, amino, alkylamino, dialkylamino and sulfonamido, then E cannot be a substituted aryl wherein two substituents are identically selected from hydroxyl and alkoxy.

In an embodiment, E is an aryl substituted with one or more of halogen, alkyl, haloalkyl, cyano, cyanoalkyl, nitro, hydroxyl, alkoxy, formyloxy, hydroxyalkyl, aldehydo, amino, alkylamino, dialkylamino, aminoalkyl, alkylaminoalkyl, mercapto, alkylmercapto, azido, and substituted or unsubstituted groups selected from alkylsulfonyl, alkylsulfinyl, alkylsulfinyloxy, alkylsulfonyloxy, alkylcarbonyloxy, carbamate, carbamido, alkoxycarbonyl, alkylaminocarbonyl, aminocarbonyl, and sulfonamido or any two substituted positions may be occupied by an alkylenedioxy group.

In accordance with an embodiment, the compound of formula (I) has the formula (II):

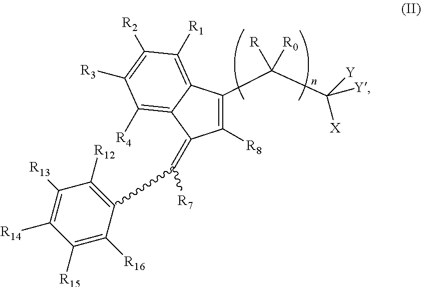

wherein:

R and R₀ are, independently, hydrogen or hydroxyl; n is 0, 1 or 2;

Y and Y′ together is oxygen;

three of R₁, R₂, R₃, and R₄ are hydrogens, and one is halogen, alkyl, or alkoxy, or two of R₁, R₂, R₃, and R₄ are hydrogens and two are alkoxy;

R₇ is hydrogen and R₈ is alkyl;

at least one of R₁₂, R₁₃, R₁₄, R₁₅, and R₁₆ is independently selected from halogen, alkyl, haloalkyl, cyano, cyanoalkyl, nitro, hydroxyl, alkoxy, formyloxy, hydroxyalkyl, aldehydo, amino, alkylamino, aminoalkyl, alkylaminoalkyl, dialkylamino, mercapto, alkylmercapto, azido, and substituted or unsubstituted groups selected from alkylsulfonyl, alkylsulfinyl, alkylsulfinyloxy, alkylsulfonyloxy, alkylcarbonyloxy, carbamate, carbamido, alkoxycarbonyl, alkylaminocarbonyl, aminocarbonyl, and sulfonamido, or any two of R₁₂, R₁₃, R₁₄, R₁₅ and R₁₆ form an alkylenedioxy group; and

X is NR′R″, where R″ is hydrogen; R′ is aryl substituted with one or more of halogen, alkyl, haloalkyl, cyano, cyanoalkyl, nitro, hydroxyl, alkoxy, formyloxy, hydroxyalkyl, aldehydo, amino, alkylamino, dialkylamino, aminoalkyl, alkylaminoalkyl, mercapto, and alkylmercapto, azido, and substituted or unsubstituted groups selected from alkylsulfonyl, alkylsulfinyl, alkylsulfinyloxy, alkylsulfonyloxy, alkylcarbonyloxy, carbamate, carbamido, alkoxycarbonyl, alkylaminocarbonyl, aminocarbonyl, and sulfonamido, or any two substituted positions may be occupied by an alkylenedioxy group;

or a pharmaceutically acceptable salt, or a prodrug thereof, or a corresponding Z or E-isomer.

In one aspect, when R and R₀ are hydrogen, n is 1, three of R₁, R₂, R₃, and R₄ are hydrogens and one is halogen, alkyl, or alkoxy or two of R₁, R₂, R₃ and R₄ are hydrogens and two are alkoxy, R₅ and R₆ together form a carbon-carbon bond, R₇ is hydrogen, R₈ is alkyl, Y and Y′ together is oxygen, X is NR′R″ wherein R″ is hydrogen, and R′ is a substituted aryl, then each of R₁₂, R₁₃, R₁₄, R₁₅ and R₁₆ must be hydrogen. For example, when R and R₀ are hydrogen, n is 1, three of R₁, R₂, R₃, and R₄ are hydrogens and one is halogen, alkyl, or alkoxy or two of R₁, R₂, R₃ and R₄ are hydrogens and two are alkoxy, R₅ and R₆ together form a carbon-carbon bond, R₇ is hydrogen, R₈ is alkyl, Y and Y′ together is oxygen, X is NR′R″ wherein R″ is hydrogen, and R′ is an aryl substituted with any of halo, alkoxy, amino, alkylamino, dialkylamino and sulfonamido, then no two of R₁₂, R₁₃, R₁₄, R₁₅ and R₁₆ can be identically selected from hydroxyl and alkoxy.

In any of the embodiments, R′ is phenyl or biphenyl substituted with one or more of halogen, alkyl, haloalkyl, cyano, cyanoalkyl, nitro, hydroxyl, alkoxy, formyloxy, hydroxyalkyl, aldehydo, amino, alkylamino, dialkylamino, aminoalkyl, alkylaminoalkyl, mercapto, and alkylmercapto, azido, and substituted or unsubstituted groups selected from alkylsulfonyl, alkylsulfinyl, alkylsulfinyloxy, alkylsulfonyloxy, alkylcarbonyloxy, carbamate, carbamido, alkoxycarbonyl, alkylaminocarbonyl, aminocarbonyl, and sulfonamido, or any two substituted positions may be occupied by an alkylenedioxy group; and at least one of R₁₂, R₁₃, R₁₄, R₁₅ and R₁₆ is independently selected from halogen, alkyl, haloalkyl, cyano, cyanoalkyl, nitro, hydroxyl, alkoxy, formyloxy, hydroxyalkyl, aldehydo, amino, alkylamino, aminoalkyl, alkylaminoalkyl, dialkylamino, mercapto, alkylmercapto, azido, and substituted or unsubstituted groups selected from alkylsulfonyl, alkylsulfinyl, alkylsulfinyloxy, alkylsulfonyloxy, alkylcarbonyloxy, carbamate, carbamido, alkoxycarbonyl, alkylaminocarbonyl, aminocarbonyl, and sulfonamido, or any two of R₁₂, R₁₃, R₁₄, R₁₅ and R₁₆ form an alkylenedioxy group.

In a particular embodiment, R′ is phenyl substituted with one or more of halogen, alkyl, trifluoromethyl, and alkoxy; and one or more of R₁₂, R₁₃, R₁₄, R₁₅ and R₁₆ is independently selected from halogen, alkyl, formyloxy, alkylcarbonyloxy, substituted or unsubstituted groups selected from alkylsulfonyl, alkylsulfinyl, alkylsulfinyloxy, alkylsulfonyloxy, carbamate, carbamido, alkoxycarbonyl, alkylaminocarbonyl, aminocarbonyl, and sulfonamido.

Examples of compounds in accordance with embodiments of the invention include:
(Z)-2-(1-(4-hydroxy-3,5-dimethoxybenzylidene)-5-methoxy-2-methyl-1H-inden-3-yl)-N-(m-tolyl)acetamide (036),
(Z)-2-(5-fluoro-1-(4-hydroxy-3,5-dimethoxybenzylidene)-2-methyl-1H-inden-3-yl)-N-(m-tolyl)acetamide (037),
(Z)—N-(3,4-dimethylphenyl)-2-(1-(4-hydroxy-3,5-dimethoxybenzylidene)-5-methoxy-2-methyl-1H-inden-3-yl)acetamide (038),
(Z)—N-(3,4-dimethylphenyl)-2-(5-fluoro-1-(4-hydroxy-3,5-dimethoxybenzylidene)-2-methyl-1H-inden-3-yl)acetamide (039),
(Z)-2-(1-(4-hydroxy-3,5-dimethoxybenzylidene)-5-methoxy-2-methyl-1H-inden-3-yl)-N-(p-tolyl)acetamide (040),
(Z)-2-(5-fluoro-1-(4-hydroxy-3,5-dimethoxybenzylidene)-2-methyl-1H-inden-3-yl)-N-(p-tolyl)acetamide (041),
(Z)-2-(1-(4-hydroxy-3,5-dimethoxybenzylidene)-5-methoxy-2-methyl-1H-inden-3-yl)-N-(o-tolyl)acetamide (042),
(Z)-2-(5-fluoro-1-(4-hydroxy-3,5-dimethoxybenzylidene)-2-methyl-1H-inden-3-yl)-N-(o-tolyl)acetamide (043),
(Z)-2-(1-(4-hydroxy-3-methoxybenzylidene)-5-methoxy-2-methyl-1H-inden-3-yl)-N-(m-tolyl)acetamide (046),
(Z)-2-(5-fluoro-1-(4-hydroxy-3-methoxybenzylidene)-2-methyl-1H-inden-3-yl)-N-(m-tolyl)acetamide (047),
(Z)-2-(1-(4-hydroxy-3-methoxybenzylidene)-5-methoxy-2-methyl-1H-inden-3-yl)-N-(3-methoxyphenyl)acetamide (048),
(Z)-2-(5-fluoro-1-(4-hydroxy-3-methoxybenzylidene)-2-methyl-1H-inden-3-yl)-N-(3-methoxyphenyl)acetamide (049),
(Z)-2-(5-fluoro-1-(4-hydroxy-3,5-dimethoxybenzylidene)-2-methyl-1H-inden-3-yl)-N-(7-methylnaphthalen-2-yl)acetamide (050),
(Z)-4-((5-fluoro-2-methyl-3-(2-oxo-2-(m-tolylamino)ethyl)-1H-inden-1-ylidene)methyl)-2,6-dimethoxyphenyl ethylcarbamate (051),
(Z)-4-((5-fluoro-2-methyl-3-(2-oxo-2-(m-tolylamino)ethyl)-1H-inden-1-ylidene)methyl)-2,6-dimethoxyphenyl methanesulfonate (052),
(Z)-2-(5-fluoro-1-(4-acetoxy-3,5-dimethoxybenzylidene)-2-methyl-1H-inden-3-yl)-N-(m-tolyl)acetamide (053), and
(Z)—N-(4-(tert-butyl)phenyl)-2-(5-fluoro-1-(4-hydroxy-3,5-dimethoxybenzylidene)-2-methyl-1H-inden-3-yl)acetamide (059), or

a pharmaceutically acceptable salt or prodrug thereof, or the corresponding E-isomer thereof.

The structural formulas of the above compounds are as follows:

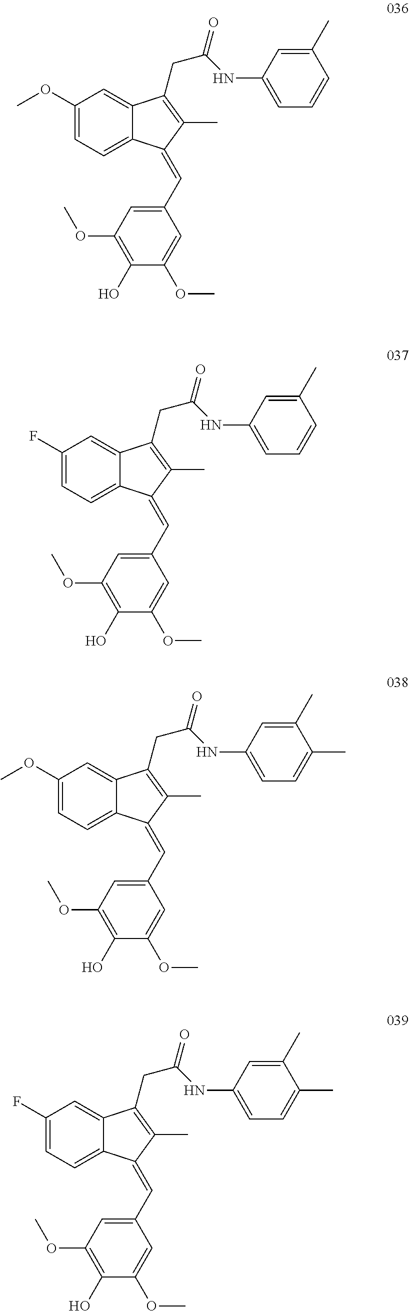

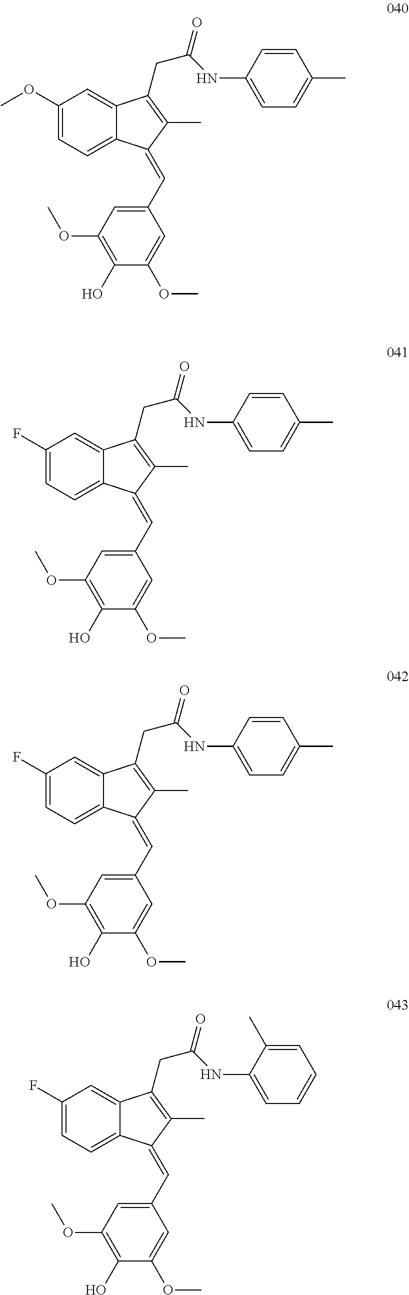

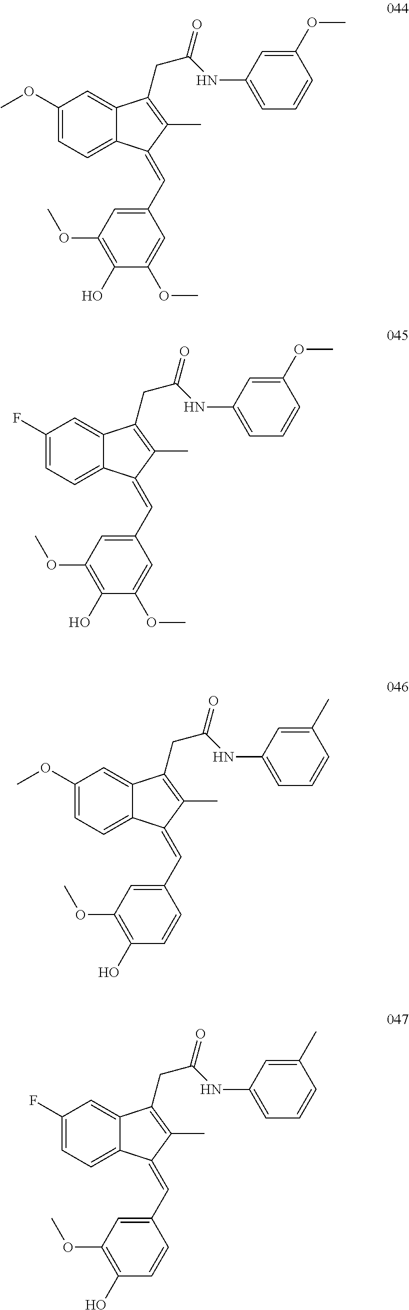

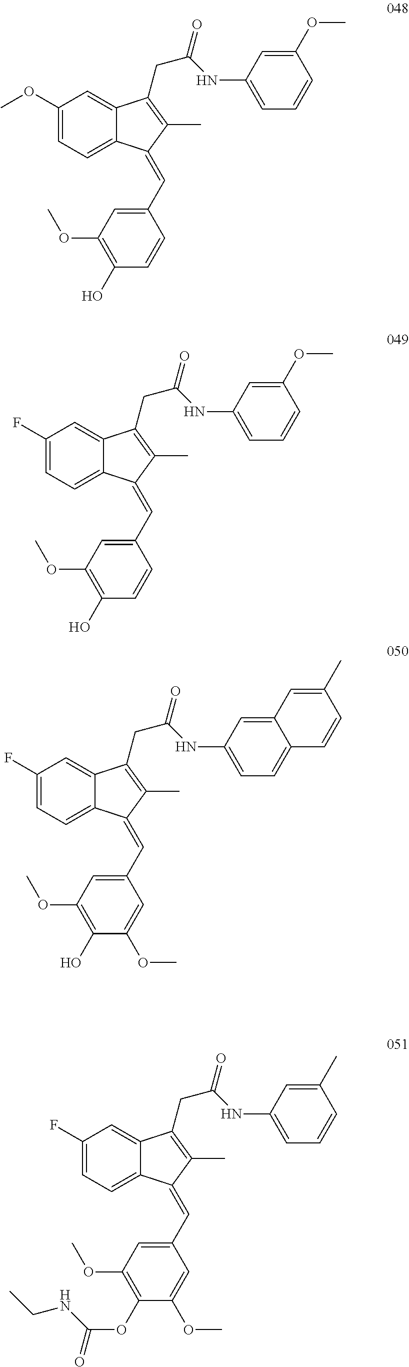

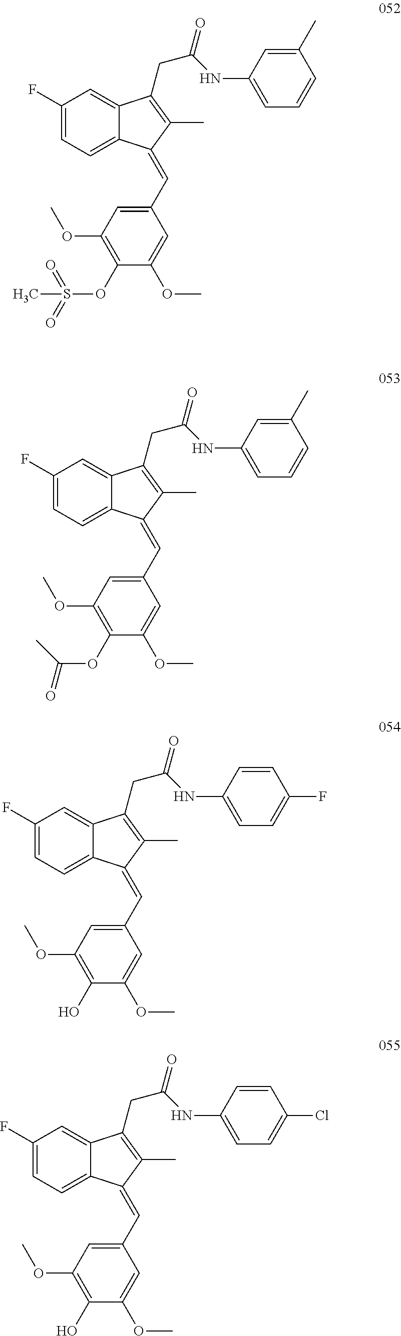 [SMILES: COc1cc(/C=C2/C(C)=C(CC(=O)Nc3cccc(C)c3)c3cc(F)ccc32)cc(OC)c1OS(C)(=O)=O ; COc1cc(/C=C2/C(C)=C(CC(=O)Nc3cccc(C)c3)c3cc(F)ccc32)cc(OC)c1OC(C)=O ; COc1cc(/C=C2/C(C)=C(CC(=O)Nc3ccc(F)cc3)c3cc(F)ccc32)cc(OC)c1O ; COc1cc(/C=C2/C(C)=C(CC(=O)Nc3ccc(Cl)cc3)c3cc(F)ccc32)cc(OC)c1O]

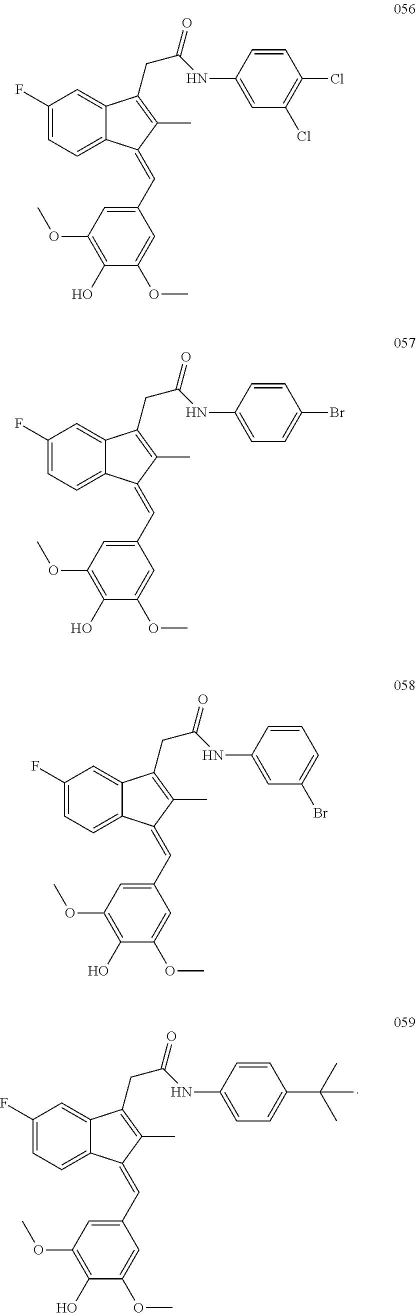 [SMILES: COc1cc(/C=C2/C(C)=C(CC(=O)Nc3ccc(Cl)c(Cl)c3)c3cc(F)ccc32)cc(OC)c1O ; COc1cc(/C=C2/C(C)=C(CC(=O)Nc3ccc(Br)cc3)c3cc(F)ccc32)cc(OC)c1O ; COc1cc(/C=C2/C(C)=C(CC(=O)Nc3cccc(Br)c3)c3cc(F)ccc32)cc(OC)c1O ; COc1cc(/C=C2/C(C)=C(CC(=O)Nc3ccc(C(C)(C)C)cc3)c3cc(F)ccc32)cc(OC)c1O]

The present invention further provides a pharmaceutical composition comprising a compound as described above, a pharmaceutically acceptable salt or prodrug thereof, or the corresponding Z or E-isomer thereof, and a pharmaceutically acceptable carrier.

In an embodiment, the pharmaceutical composition further includes at least one additional therapeutic agent other than a compound of formula I, a pharmaceutically acceptable salt or prodrug thereof, or the corresponding Z or E-isomer thereof.

The present invention further provides a method of inhibiting a human or nonhuman mammalian Ras-mediated biological process, which method comprising administering in vivo or in vitro a Ras-inhibitory amount of at least one compound of formula I, a pharmaceutically acceptable salt or prodrug thereof:

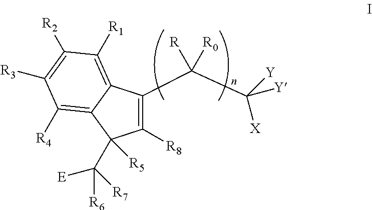

wherein:

R and R₀ are, independently, hydrogen or hydroxyl; n is 0, 1 or 2;

three of R₁, R₂, R₃, and R₄ are hydrogens, and one is halogen, alkyl, or alkoxy, or two of R₁, R₂, R₃, and R₄ are hydrogens and two are alkoxy;

R₅ and R₆ together form a carbon-carbon bond; R₇ is hydrogen; R₈ is alkyl;

Y and Y′ together is oxygen;

X is NR′R″, where R′ is aryl substituted with one or more of halogen, alkyl, haloalkyl, cyano, cyanoalkyl, nitro, hydroxyl, alkoxy, formyloxy, hydroxyalkyl, aldehydo, amino, alkylamino, dialkylamino, aminoalkyl, alkylaminoalkyl, mercapto, and alkylmercapto, azido, and substituted or unsubstituted groups selected from alkylsulfonyl, alkylsulfinyl, alkylsulfinyloxy, alkylsulfonyloxy, alkylcarbonyloxy, carbamate, carbamido, alkoxycarbonyl, alkylaminocarbonyl, aminocarbonyl, and sulfonamido, or any two substituted positions may be occupied by an alkylenedioxy group; and R″ is hydrogen; and

E is a substituted aryl;

wherein the compound of formula I or pharmaceutically acceptable salt or prodrug thereof or Z or E-isomer is administered alone or in combination with at least one additional therapeutic agent other than said compound of formula I or pharmaceutically acceptable salt or prodrug thereof or Z or E-isomer.

The present invention further provides a method of therapeutically or prophylactically treating a human or nonhuman mammalian patient with a disease or condition treatable by inhibition of one or more Ras-mediated biological process, which method comprising administering to a patient in need thereof a therapeutically or prophylactically effective amount of at least one ras-inhibitory compound of formula I, a pharmaceutically acceptable salt or prodrug thereof:

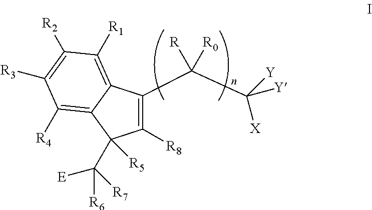

wherein:

R and R₀ are, independently, hydrogen or hydroxyl; n is 0, 1 or 2;

three of R₁, R₂, R₃, and R₄ are hydrogens, and one is halogen, alkyl, or alkoxy, or two of R₁, R₂, R₃, and R₄ are hydrogens and two are alkoxy;

R₅ and R₆ together form a carbon-carbon bond; R₇ is hydrogen; R₈ is alkyl;

Y and Y′ together is oxygen;

X is NR′R″, where R′ is aryl substituted with one or more of halogen, alkyl, haloalkyl, cyano, cyanoalkyl, nitro, hydroxyl, alkoxy, formyloxy, hydroxyalkyl, aldehydo, amino, alkylamino, dialkylamino, aminoalkyl, alkylaminoalkyl, mercapto, and alkylmercapto, azido, and substituted or unsubstituted groups selected from alkylsulfonyl, alkylsulfinyl, alkylsulfinyloxy, alkylsulfonyloxy, alkylcarbonyloxy, carbamate, carbamido, alkoxycarbonyl, alkylaminocarbonyl, aminocarbonyl, and sulfonamido, or any two substituted positions may be occupied by an alkylenedioxy group; and R″ is hydrogen; and

E is a substituted aryl;

wherein the compound of formula I or pharmaceutically acceptable salt or prodrug thereof or Z or E-isomer is administered alone or in combination with at least one additional therapeutic agent other than said compound of formula I or pharmaceutically acceptable salt or prodrug thereof or Z or E-isomer.

In any of the methods above, the E of the compound is an aryl substituted with one or more of halogen, alkyl, haloalkyl, cyano, cyanoalkyl, nitro, hydroxyl, alkoxy, formyloxy, hydroxyalkyl, aldehydo, amino, alkylamino, dialkylamino, aminoalkyl, alkylaminoalkyl, mercapto, and alkylmercapto, azido, and substituted or unsubstituted groups selected from alkylsulfonyl, alkylsulfinyl, alkylsulfinyloxy, alkylsulfonyloxy, alkylcarbonyloxy, carbamate, carbamido, alkoxycarbonyl, alkylaminocarbonyl, aminocarbonyl, and sulfonamido or any two substituted positions may be occupied by an alkylenedioxy group.

In one aspect of the method above, when R and R₀ are hydrogen, n is 1, three of R₁, R₂, R₃, and R₄ are hydrogens and one is halogen, alkyl, or alkoxy or two of R₁, R₂, R₃ and R₄ are hydrogens and two are alkoxy, R₅ and R₆ together form a carbon-carbon bond, R₇ is hydrogen, R₈ is alkyl, Y and Y′ together is oxygen, X is NR′R″ wherein R″ is hydrogen, and R′ is a substituted aryl, then E cannot be a substituted aryl. For example, when R and R₀ are hydrogen, n is 1, three of R₁, R₂, R₃, and R₄ are hydrogens and one is halogen, alkyl, or alkoxy or two of R₁, R₂, R₃ and R₄ are hydrogens and two are alkoxy, R₅ and R₆ together form a carbon-carbon bond, R₇ is hydrogen, R₈ is alkyl, Y and Y′ together is oxygen, X is NR′R″ wherein R″ is hydrogen, R′ is an aryl substituted with any of halo, alkoxy, amino, alkylamino, dialkylamino and sulfonamido, then E cannot be a substituted aryl wherein two substituents are identically selected from hydroxyl and alkoxy.

The present invention further provides a method of inhibiting a human or nonhuman mammalian Ras-mediated biological process, which method comprising administering in vivo or in vitro a Ras-inhibitory amount of at least one compound selected from:
(Z)-2-(1-(4-hydroxy-3,5-dimethoxybenzylidene)-5-methoxy-2-methyl-1H-inden-3-yl)-N-(m-tolyl)acetamide (036),
(Z)-2-(5-fluoro-1-(4-hydroxy-3,5-dimethoxybenzylidene)-2-methyl-1H-inden-3-yl)-N-(m-tolyl)acetamide (037),
(Z)—N-(3,4-dimethylphenyl)-2-(1-(4-hydroxy-3,5-dimethoxybenzylidene)-5-methoxy-2-methyl-1H-inden-3-yl)acetamide (038),
(Z)—N-(3,4-dimethylphenyl)-2-(5-fluoro-1-(4-hydroxy-3,5-dimethoxybenzylidene)-2-methyl-1H-inden-3-yl)acetamide (039),
(Z)-2-(1-(4-hydroxy-3,5-dimethoxybenzylidene)-5-methoxy-2-methyl-1H-inden-3-yl)-N-(p-tolyl)acetamide (040),
(Z)-2-(5-fluoro-1-(4-hydroxy-3,5-dimethoxybenzylidene)-2-methyl-1H-inden-3-yl)-N-(p-tolyl)acetamide (041),
(Z)-2-(1-(4-hydroxy-3,5-dimethoxybenzylidene)-5-methoxy-2-methyl-1H-inden-3-yl)-N-(o-tolyl)acetamide (042),
(Z)-2-(5-fluoro-1-(4-hydroxy-3,5-dimethoxybenzylidene)-2-methyl-1H-inden-3-yl)-N-(o-tolyl)acetamide (043),
(Z)-2-(1-(4-hydroxy-3,5-dimethoxybenzylidene)-5-methoxy-2-methyl-1H-inden-3-yl)-N-(3-methoxyphenyl)acetamide (044),
(Z)-2-(5-fluoro-1-(4-hydroxy-3,5-dimethoxybenzylidene)-2-methyl-1H-inden-3-yl)-N-(3-methoxyphenyl)acetamide (045),
(Z)-2-(1-(4-hydroxy-3-methoxybenzylidene)-5-methoxy-2-methyl-1H-inden-3-yl)-N-(m-tolyl)acetamide (046),
(Z)-2-(5-fluoro-1-(4-hydroxy-3-methoxybenzylidene)-2-methyl-1H-inden-3-yl)-N-(m-tolyl)acetamide (047),
(Z)-2-(1-(4-hydroxy-3-methoxybenzylidene)-5-methoxy-2-methyl-1H-inden-3-yl)-N-(3-methoxyphenyl)acetamide (048),
(Z)-2-(5-fluoro-1-(4-hydroxy-3-methoxybenzylidene)-2-methyl-1H-inden-3-yl)-N-(3-methoxyphenyl)acetamide (049),
(Z)-2-(5-fluoro-1-(4-hydroxy-3,5-dimethoxybenzylidene)-2-methyl-1H-inden-3-yl)-N-(7-methylnaphthalen-2-yl)acetamide (050),
(Z)-4-((5-fluoro-2-methyl-3-(2-oxo-2-(m-tolylamino)ethyl)-1H-inden-1-ylidene)methyl)-2,6-dimethoxyphenyl ethylcarbamate (051),
(Z)-4-((5-fluoro-2-methyl-3-(2-oxo-2-(m-tolylamino)ethyl)-1H-inden-1-ylidene)methyl)-2,6-dimethoxyphenyl methanesulfonate (052),
(Z)-2-(5-fluoro-1-(4-acetoxy-3,5-dimethoxybenzylidene)-2-methyl-1H-inden-3-yl)-N-(m-tolyl)acetamide (053),
(Z)-2-(5-fluoro-1-(4-hydroxy-3,5-dimethoxybenzylidene)-2-methyl-1H-inden-3-yl)-N-(4-fluorophenyl)acetamide (054),
(Z)—N-(4-chlorophenyl)-2-(5-fluoro-1-(4-hydroxy-3,5-dimethoxybenzylidene)-2-methyl-1H-inden-3-yl)acetamide (055),
(Z)—N-(3,4-dichlorophenyl)-2-(5-fluoro-1-(4-hydroxy-3,5-dimethoxybenzylidene)-2-methyl-1H-inden-3-yl)acetamide (056),
(Z)—N-(4-bromophenyl)-2-(5-fluoro-1-(4-hydroxy-3,5-dimethoxybenzylidene)-2-methyl-1H-inden-3-yl)acetamide (057),
(Z)—N-(3-bromophenyl)-2-(5-fluoro-1-(4-hydroxy-3,5-dimethoxybenzylidene)-2-methyl-1H-inden-3-yl)acetamide (058), and
(Z)—N-(4-(tert-butyl)phenyl)-2-(5-fluoro-1-(4-hydroxy-3,5-dimethoxybenzylidene)-2-methyl-1H-inden-3-yl)acetamide (059), or

a pharmaceutically acceptable salt or prodrug thereof, or the corresponding E-isomer thereof,

wherein said compound, pharmaceutically acceptable salt or prodrug thereof or E-isomer thereof, is administered alone or in combination with at least one additional therapeutic agent other than said compound, pharmaceutically acceptable salt, prodrug, or E-isomer thereof.

The present invention further provides a method of therapeutically or prophylactically treating a human or nonhuman mammalian patient with a disease or condition treatable by inhibition of one or more Ras-mediated biological process, which method comprising administering to a patient in need thereof a therapeutically or prophylactically effective amount of at least one ras-inhibitory compound selected from:
(Z)-2-(1-(4-hydroxy-3,5-dimethoxybenzylidene)-5-methoxy-2-methyl-1H-inden-3-yl)-N-(m-tolyl)acetamide (036),
(Z)-2-(5-fluoro-1-(4-hydroxy-3,5-dimethoxybenzylidene)-2-methyl-1H-inden-3-yl)-N-(m-tolyl)acetamide (037),
(Z)—N-(3,4-dimethylphenyl)-2-(1-(4-hydroxy-3,5-dimethoxybenzylidene)-5-methoxy-2-methyl-1H-inden-3-yl)acetamide (038),
(Z)—N-(3,4-dimethylphenyl)-2-(5-fluoro-1-(4-hydroxy-3,5-dimethoxybenzylidene)-2-methyl-1H-inden-3-yl)acetamide (039),
(Z)-2-(1-(4-hydroxy-3,5-dimethoxybenzylidene)-5-methoxy-2-methyl-1H-inden-3-yl)-N-(p-tolyl)acetamide (040),
(Z)-2-(5-fluoro-1-(4-hydroxy-3,5-dimethoxybenzylidene)-2-methyl-1H-inden-3-yl)-N-(p-tolyl)acetamide (041),
(Z)-2-(1-(4-hydroxy-3,5-dimethoxybenzylidene)-5-methoxy-2-methyl-1H-inden-3-yl)-N-(o-tolyl)acetamide (042),
(Z)-2-(5-fluoro-1-(4-hydroxy-3,5-dimethoxybenzylidene)-2-methyl-1H-inden-3-yl)-N-(o-tolyl)acetamide (043),
(Z)-2-(1-(4-hydroxy-3,5-dimethoxybenzylidene)-5-methoxy-2-methyl-1H-inden-3-yl)-N-(3-methoxyphenyl)acetamide (044),
(Z)-2-(5-fluoro-1-(4-hydroxy-3,5-dimethoxybenzylidene)-2-methyl-1H-inden-3-yl)-N-(3-methoxyphenyl)acetamide (045),
(Z)-2-(1-(4-hydroxy-3-methoxybenzylidene)-5-methoxy-2-methyl-1H-inden-3-yl)-N-(m-tolyl)acetamide (046),
(Z)-2-(5-fluoro-1-(4-hydroxy-3-methoxybenzylidene)-2-methyl-1H-inden-3-yl)-N-(m-tolyl)acetamide (047),
(Z)-2-(1-(4-hydroxy-3-methoxybenzylidene)-5-methoxy-2-methyl-1H-inden-3-yl)-N-(3-methoxyphenyl)acetamide (048),
(Z)-2-(5-fluoro-1-(4-hydroxy-3-methoxybenzylidene)-2-methyl-1H-inden-3-yl)-N-(3-methoxyphenyl)acetamide (049),
(Z)-2-(5-fluoro-1-(4-hydroxy-3,5-dimethoxybenzylidene)-2-methyl-1H-inden-3-yl)-N-(7-methylnaphthalen-2-yl)acetamide (050),
(Z)-4-((5-fluoro-2-methyl-3-(2-oxo-2-(m-tolylamino)ethyl)-1H-inden-1-ylidene)methyl)-2,6-dimethoxyphenyl ethylcarbamate (051),
(Z)-4-((5-fluoro-2-methyl-3-(2-oxo-2-(m-tolylamino)ethyl)-1H-inden-1-ylidene)methyl)-2,6-dimethoxyphenyl methanesulfonate (052),
(Z)-2-(5-fluoro-1-(4-acetoxy-3,5-dimethoxybenzylidene)-2-methyl-1H-inden-3-yl)-N-(m-tolyl)acetamide (053),
(Z)-2-(5-fluoro-1-(4-hydroxy-3,5-dimethoxybenzylidene)-2-methyl-1H-inden-3-yl)-N-(4-fluorophenyl)acetamide (054),
(Z)—N-(4-chlorophenyl)-2-(5-fluoro-1-(4-hydroxy-3,5-dimethoxybenzylidene)-2-methyl-1H-inden-3-yl)acetamide (055),
(Z)—N-(3,4-dichlorophenyl)-2-(5-fluoro-1-(4-hydroxy-3,5-dimethoxybenzylidene)-2-methyl-1H-inden-3-yl)acetamide (056),
(Z)—N-(4-bromophenyl)-2-(5-fluoro-1-(4-hydroxy-3,5-dimethoxybenzylidene)-2-methyl-1H-inden-3-yl)acetamide (057),
(Z)—N-(3-bromophenyl)-2-(5-fluoro-1-(4-hydroxy-3,5-dimethoxybenzylidene)-2-methyl-1H-inden-3-yl)acetamide (058), and
(Z)—N-(4-(tert-butyl)phenyl)-2-(5-fluoro-1-(4-hydroxy-3,5-dimethoxybenzylidene)-2-methyl-1H-inden-3-yl)acetamide (059), or

a pharmaceutically acceptable salt or prodrug thereof, or the corresponding E-isomer thereof,

wherein said compound, pharmaceutically acceptable salt or prodrug thereof, or E-isomer thereof, is administered alone or in combination with at least one additional therapeutic agent other than said compound, pharmaceutically acceptable salt, prodrug, or E-isomer thereof.

In any of the embodiments of the methods above, the Ras-mediated biological process is selected from growth, proliferation, survival, metastasis, drug resistance and radiation resistance of a tumor cell.

In an embodiment of the methods, the Ras-mediated biological process is cancer, for example, a cancer selected from pancreatic cancer, lung cancer, colorectal cancer, melanoma, ovarian cancer, renal cancer, prostate cancer, head and neck cancer, endocrine cancer, uterine cancer, breast cancer, sarcoma cancer, gastric cancer, hepatic cancer, esophageal cancer, central nervous system cancer, brain cancer, hepatic cancer, germline cancer, lymphoma, and leukemia, and particularly selected from pancreatic cancer, colorectal cancer, and lung cancer.

In any of the embodiments, the cancer is drug-resistant or radiation-resistant.

In any of the embodiments, the patient is pre-selected by utilizing an assay of said patient's tissue, blood or tumor for an abnormal, mutant or hyperactive ras gene or Ras protein, or an aberrant Ras-mediated biological process.

In any of the embodiments, the patient's tissue, blood or tumor contains an abnormal, mutant or hyperactive ras gene or Ras protein, or aberrant Ras-mediated biological process.

The present invention further provides a method of therapeutically or prophylactically treating a human or non-human mammalian patient with cancer, which method comprises administering to a patient in need thereof a therapeutically or prophylactically effective amount of at least one compound according to formula I or II, or pharmaceutically acceptable salt or prodrug thereof, or Z or E-isomer thereof, wherein the compound, pharmaceutically acceptable salt or prodrug thereof, or Z or E-isomer thereof, is administered alone or in combination with at least one additional therapeutic agent other than the above compound, pharmaceutically acceptable salt, prodrug, or Z or E-isomer thereof.

The present invention further provides a method of therapeutically or prophylactically treating a human or nonhuman mammalian patient with a disease or condition treatable by the inhibition of one or more neoplastic or cancerous process, which method comprises administering to a patient in need thereof a therapeutically or prophylactically effective amount of at least one neoplastic or cancerous process inhibitory compound according to formula I or II, or pharmaceutically acceptable salt or prodrug thereof, or Z or E-isomer thereof, wherein said compound, pharmaceutically acceptable salt or prodrug thereof, or Z or E-isomer thereof is administered alone or in combination with at least one additional therapeutic agent other than the above compound, pharmaceutically acceptable salt, prodrug, or Z or E-isomer thereof.

In an embodiment, the neoplastic or cancerous process is a Ras-mediated biological process is selected from growth, proliferation, survival, metastasis, drug resistance and radiation resistance of a tumor cell.

In an embodiment, the cancer is treatable by the inhibition of one or more Ras-mediated biological process.

In an embodiment, the Ras-mediated biological process is cancer, for example, a cancer selected from pancreatic cancer, lung cancer, colorectal cancer, melanoma, ovarian cancer, renal cancer, prostate cancer, head and neck cancer, endocrine cancer, uterine cancer, breast cancer, sarcoma cancer, gastric cancer, hepatic cancer, esophageal cancer, central nervous system cancer, brain cancer, hepatic cancer, germline cancer, lymphoma, and leukemia, particularly selected from pancreatic cancer, colorectal cancer, and lung cancer.

In any of the embodiments, the cancer is drug-resistant or radiation-resistant.

The present invention further provides a pharmaceutical composition comprising a compound as described above, a pharmaceutically acceptable salt or prodrug thereof, and a pharmaceutically acceptable carrier.

In accordance with an embodiment, the pharmaceutical composition may further include at least one additional therapeutic agent other than a compound of formulas I-II.

In an embodiment, the present invention provides a method of therapeutically or prophylactically treating a human or nonhuman mammalian patient with cancer, which method comprises administering to a patient in need thereof a therapeutically or prophylactically effective amount of at least one compound of formula I or II.

In an embodiment, the cancer is selected from pancreatic cancer, lung cancer, colorectal cancer, melanoma, ovarian cancer, renal cancer, prostate cancer, head and neck cancer, endocrine cancer, uterine cancer, breast cancer, sarcoma cancer, gastric cancer, hepatic cancer, esophageal cancer, central nervous system cancer, brain cancer, hepatic cancer, germline cancer, lymphoma, and leukemia, preferably pancreatic cancer, colorectal cancer, or lung cancer. In an embodiment, the cancer is drug-resistant or radiation-resistant.

In a preferred embodiment of the above method, the neoplastic or cancerous process is selected from growth, proliferation, survival, metastasis, drug resistance and radiation resistance of a tumor cell.

In an embodiment, the compounds of the invention include Ras-inhibitory compounds. A Ras-inhibitory compound can be identified from one or more compounds of formulas I-II by an assay of Ras inhibition. Some representative assays of selective Ras inhibition are illustrated in the examples that follow herein. As used herein, the terminology selective “Ras inhibition” means selective, preferential or specific inhibition of aberrant Ras-mediated cellular processes, such as, for example, accelerated or aberrant cell growth, proliferation, survival, and invasiveness, relative to these processes in cells or tissues with normal or non-aberrant Ras and Ras-mediated processes. Experimentally, selective Ras inhibition can be shown, for example, by determining the ratio (numerator/denominator) of a given compound's potency (e.g., IC₅₀) to inhibit the growth of cells with “normal” or “wild-type” Ras (numerator) relative to that of cells with mutated and/or activated Ras (denominator). The terminology used herein for such an experimentally determined ratio is “selectivity” or “selectivity index”, which may be further denoted by showing the respective cell types used to determine the numerical ratio (e.g., HT-29/A549; Caco-2/SW-480; HT-29/SW-480; HT-29/CCT-116). For a given compound, a “selectivity” value or “selectivity index” of greater than 1 (one), preferably greater than 10 (ten), more preferably greater than 100 (one hundred) and even more preferably greater than 1000 (one thousand) indicates said compound selectively inhibits hyperactive Ras and/or Ras-mediated cellular functions, such as those which may drive or accelerate cancer cell growth, proliferation, metastasis, resistance to drugs or radiation, and the like.

In another preferred embodiment of the present invention, the aforementioned assay of Ras inhibition employs one or more isogenic cell line pair(s), wherein both of the lines share the same genetic background except that one of the lines (“mutant line”) contains one or more mutated or hyperactive ras gene(s), Ras protein(s) and/or aberrant Ras-mediated biological process(es), and the other line (“normal line”) lacks such mutation(s) or aberrant function(s).

In a further preferred embodiment of the present invention, the aforementioned assay employing isogenic cell line(s) enables the determination and calculation of a Ras-Inhibitory Specificity Index (RISI). One experimental approach to determination of such a RISI may, for example, comprise determining the ratio of the concentration of a compound producing a specified effect on the normal line, such as, for example, 50% growth inhibition in a specified period of time, divided by the concentration of the same compound producing the same specified effect (e.g., 50% growth inhibition in the same specified period of time) on the mutant line.

Whereas in the aforementioned approach, the 50% growth inhibition values may be obtained by testing the compound against both normal and mutant cell lines at multiple concentrations over a specified concentration range, for example 10 nM-10,000 nM, an alternate, more streamlined approach to determining a RISI value could comprise measuring the ratio of percentage growth inhibition in a given period of time by a specified single concentration of the compound, for example 250 nM, selected from within a range of concentrations, for example from within a range of 10 nM-10,000 nM, against the mutant (numerator) relative to the normal cell line (denominator). This approach may be generally more applicable to larger-scale or preliminary screening of groups of individual compounds or mixtures thereof to obtain a preliminary or screening RISI, whereas a RISI determined using concentration ranges to determine 50% growth inhibition values may be more precise. A RISI value obtained for a given compound by either approach may be less than, equal to or greater than 1 (one), and a RISI value of greater than 1 (one) indicates said compound selectively inhibits Ras or Ras-mediated cellular functions.

In a highly preferred embodiment of the present invention, the employed assay of Ras inhibition enables identification of a compound from one or more compounds of formulas I-II having a RISI of greater than 1, preferably greater than 10, more preferably greater than 100, and even more preferably greater than 1000.

The present invention yet further provides a pharmaceutical composition comprising a therapeutically effective amount of Ras-inhibitory activity from one or more Ras-inhibitory compound(s) of formula I-II, or pharmaceutically acceptable salt(s) or prodrug(s) thereof, alone or in combination with at least one additional therapeutic agent. The therapeutically effective amount can be that amount provided by a Ras-inhibiting and/or a disease-process inhibiting effective amount, such as an anticancer effective amount, of a compound of formula I-II.

In addition, the present invention provides a method of therapeutically or prophylactically treating a condition treatable by the inhibition of Ras-mediated biological processes including, for example, tumor cell growth, proliferation, survival, invasion and metastasis, as well as resistance to chemotherapy, other molecularly targeted therapeutics, and radiation; and, a method of therapeutically or prophylactically treating cancers harboring hyperactive or mutant Ras. These methods comprise administering a therapeutically or prophylactically effective amount of Ras-inhibiting activity from at least one Ras-inhibitory compound, or pharmaceutically acceptable salt or prodrug thereof, of formula I-II.

For example, the disease or condition treatable by the inhibition of one or more Ras-mediated biological process is cancer, neurofibromatosis, or Costello syndrome. In an embodiment, the Ras-mediated biological process is selected from growth, proliferation, survival, metastasis, drug resistance and radiation resistance of a tumor cell.

In an embodiment, the cancer is selected from pancreatic cancer, lung cancer, colorectal cancer, melanoma, ovarian cancer, renal cancer, prostate cancer, head and neck cancer, endocrine cancer, uterine cancer, breast cancer, sarcoma cancer, gastric cancer, hepatic cancer, esophageal cancer, central nervous system cancer, brain cancer, hepatic cancer, germline cancer, lymphoma, and leukemia, particularly pancreatic cancer, colorectal cancer, and lung cancer. In accordance with an embodiment, the cancer is drug-resistant or radiation-resistant.

In an embodiment of the above method, the patient is pre-selected by utilizing an assay of the patient's tissue, blood or tumor for an abnormal, mutant or hyperactive ras gene or Ras protein, or an aberrant Ras-mediated biological process.

In an embodiment, the patient's tissue, blood or tumor contains an abnormal, mutant or hyperactive ras gene or Ras protein, or aberrant Ras-mediated biological process.

The compounds in the present invention also can be in the form of a pharmaceutically acceptable salt, which may include, for example, the salt of one or more acidic substituents (e.g. a carboxylic salt, a sulfonic acid salt, and the like) and the salt of one or more basic substituents (e.g. the salt of an amine, and the like). Suitable salts of acidic substituents include, for example, metal salts (e.g. sodium salts, potassium salts, magnesium salts, zinc salts, and the like) and ammonium salts (e.g., NH₄+ salts, alkylammonium salts, quaternary ammonium salts, and the like). Suitable salts of basic substituents include, for example, acid addition salts (e.g., hydrochloride salts, hydrobromide salts, carboxylate salts (e.g., acetate salts), sulfate salts, sulfonate salts (e.g., mesylate salts), phosphate salts, quaternary ammonium salts, and the like.

A compound of the present invention can also be provided as a prodrug, which is a drug derivative or drug precursor compound that typically is inactive or less than fully active until it is converted in the body through a normal metabolic process such as, for example, hydrolysis of an ester or amide form of the drug, to the active drug. A prodrug may be selected and used instead of the parent drug because, for example, in its prodrug form it is less toxic, and/or may have better absorption, distribution, metabolism and excretion (ADME) characteristics, and the like, than the parent drug. A prodrug might also be used to improve how selectively the drug interacts with cells or processes that are not its intended target. This approach may be employed particularly, for example, to prevent or decrease adverse effects, especially in cancer treatments, which may be especially prone to having severe unintended and undesirable side effects.

The term “prodrug” denotes a derivative of a compound, which derivative, when administered to warm-blooded animals, e.g. humans, is converted into the compound (drug). For example, the enzymatic and/or chemical hydrolytic cleavage of a derivative compound of the present invention occurs in such a manner that the proven drug form is released, and the moiety or moieties split off remain nontoxic or are metabolized so that nontoxic metabolites are produced. For example, a carboxylic acid group can be esterified, e.g., with a methyl group or ethyl group to yield an ester. When an ester is administered to a subject, the ester is cleaved, enzymatically or non-enzymatically, reductively, oxidatively, or hydrolytically, to reveal the anionic group. An anionic group can be esterified with moieties (e.g., acyloxymethyl esters) which are cleaved to reveal an intermediate compound which subsequently decomposes to yield the active compound.

The prodrugs can be prepared in situ during the isolation and purification of the compounds, or by separately reacting the purified compound with a suitable derivatizing agent. For example, hydroxy groups can be converted into esters via treatment with a carboxylic acid in the presence of a catalyst. Examples of cleavable alcohol prodrug moieties include substituted or unsubstituted, branched or unbranched alkyl ester moieties, e.g., ethyl esters, alkenyl esters, di-alkylamino alkyl esters, e.g., dimethylaminoethyl ester, acylamino alkyl esters, acyloxy alkyl esters (e.g., pivaloyloxymethyl ester), aryl esters, e.g., phenyl ester, aryl-alkyl esters, e.g., benzyl ester, optionally substituted, e.g., with methyl, halo, or methoxy substituents aryl and aryl-alkyl esters, amides, alkyl amides, di-alkyl amides, and hydroxy amides.

Knowing the disclosures herein, it will be appreciated also that a compound of the present invention can be in the form of a prodrug, and that such prodrugs can be prepared using reagents and synthetic transformations that are well-known to those having ordinary skill in the art. The effectiveness of a particular prodrug can be determined using one or more analytical methods (e.g. pharmacokinetics, bioassays, in vivo efficacy studies, and the like) that are well-known to those of ordinary skill in the art.

More specifically, a prodrug of a compound of formula I-II may be prepared using routine chemical procedures, such as the exemplary procedures described herein. For example, any of the E groups can be substituted on the ring with a group of the formula Q-U-, for example,

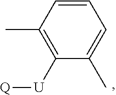

wherein U is selected from the group consisting of oxygen, sulfur, nitrogen, OCH₂, SCH₂ and NHCH₂; and Q is selected from the group consisting of PEG-CO, HCO, acetyl, amino acid, substituted benzoic acid and phosphoric acid.

Suitable prodrugs may include, but not be limited to, those illustrated below for a compound of formula I, specifically as exemplary prodrug derivatives of compound 037:

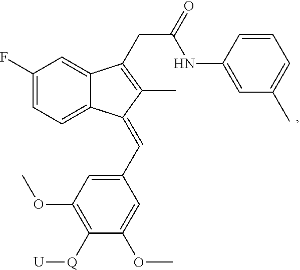

wherein U is selected from the group consisting of oxygen, sulfur, nitrogen, OCH₂, SCH₂ and NHCH₂; and Q is selected from the group consisting of PEG-CO, HCO, acetyl, amino acid, substituted benzoic acid and phosphoric acid.

Examples of prodrugs of compounds 044, 058, and 042 are illustrated below:

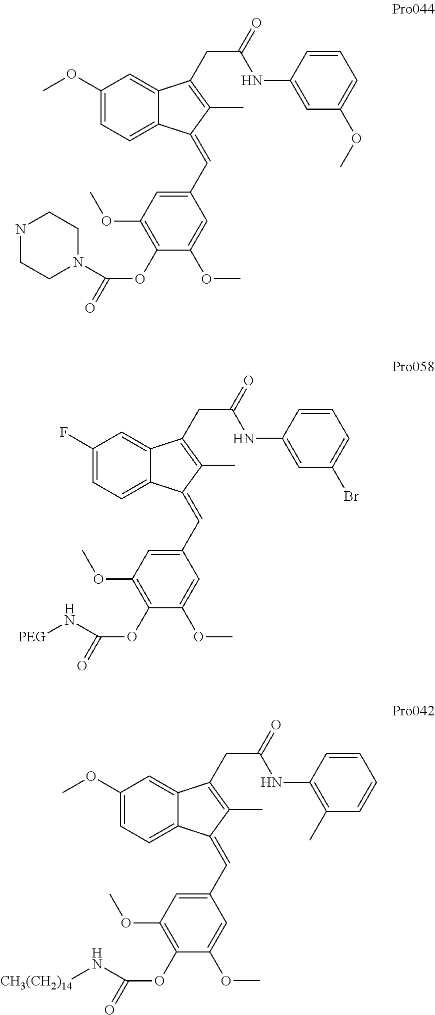

As used herein, the “alkyl” part of any of the substituents described herein, e.g., but not limited to, alkyl, alkylamino, alkylmercapto, hydroxyalkyl, polyhydroxyalkyl, alkylaminoalkyl, aminoalkyl, arylalkyl, arylcycloalkyl, heterocycloalkyl, arylalkylenyl, arylcycloalkyl, dialkylamino, alkylcarbonyloxy, dialkylaminoalkyl, cyanoalkyl, haloalkyl, alkylcarbonylalkylcarbonyloxy, dialkylalkylaminoalkyl, alkylsulfonyl, alkylsulfinyl, alkylsulfinyloxy, alkylsulfonyloxy, alkylenedioxy, carbocycloalkyl, and phenylalkyl, means a straight-chain or branched-chain saturated alkyl which can contain from 1-20 carbon atoms, for example from 1 to about 10 carbon atoms, or from 1 to about 8 carbon atoms, or, preferably, lower alkyl, i.e., from 1 to 6 carbon atoms.

Examples of alkyls include methyl, ethyl, propyl, isopropyl, n-butyl, sec-butyl, isobutyl, tert-butyl, pentyl, isoamyl, hexyl, octyl, dodecanyl, octadecyl, and the like. Alkyl substituents can be unsubstituted or substituted, for example with at least one substituent selected from the group consisting of a halogen, a nitro, an amino, a hydroxyl, a thio, an acyl, a mercapto, and a cyano.

The term “alkenyl” means a straight-chain or branched-chain alkenyl having one or more double bonds. Unless otherwise specified, the alkenyl can contain from 2 to about 10 carbon atoms, for example from 2 to about 8 carbon atoms, or preferably from 2 to about 6 carbon atoms. Examples of alkenyls include vinyl, allyl, 1,4-butadienyl, and isopropenyl substituents, and the like.

The term “alkynyl” means a straight-chain or branched-chain alkynyl having one or more triple bonds. Unless otherwise specified, alkynyls can contain from 2 to about 10 carbon atoms, for example, from 2 to about 8 carbon atoms, or preferably, from 2 to about 6 carbon atoms. Examples of alkynyls include ethynyl, propynyl (propargyl), butynyl, and the like. Alkenyl or alkynyl substituents can be unsubstituted or substituted, for example, with at least one substituent selected from the group consisting of a halogen, a nitro, an amino, a hydroxyl, a thio, an acyl, an alkyl, and a cyano.

The term “aryl” means an aromatic carbocyclic radical, as commonly understood in the art, and includes monocyclic and polycyclic aromatics such as, for example, phenyl and naphthyl rings. Preferably, the aryl comprises one or more six-membered rings including, for example, phenyl, naphthyl, biphenyl, and the like. Typically, the aryl comprises six or more carbon atoms in the ring skeleton thereof (e.g., from 6 to about 10 carbon atoms making up the ring). Unless specified otherwise, “aryl” by itself refers to unsubstituted aryl groups and does not cover substituted aryl groups. Substituted aryl can be an aryl substituted, for example, with at least one substituent selected from the group consisting of a halogen, a nitro, an amino, a hydroxyl, a thio, an acyl, and alkyl, and a cyano. It is to be noted that arylalkyl, benzyl, or heteroaryl groups are not considered “aryl” in accordance with the present invention.

The term “heterocyclyl” includes heteroraryl. In accordance with the invention, the term “heteroaryl” refers to a cyclic aromatic radical having from five to ten ring atoms of which at least one atom is O, S, or N, and the remaining atoms are carbon. Examples of heteroaryl radicals include pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, and isoquinolinyl.

Whenever a range of the number of atoms in a structure is indicated (e.g., a C₁₋₁₂, C₁₋₈, C₁₋₆, or C₁₋₄ alkyl, alkylamino, etc.), it is specifically contemplated that any sub-range or individual number of carbon atoms falling within the indicated range also can be used. Thus, for instance, the recitation of a range of 1-8 carbon atoms (e.g., C₁-C₈), 1-6 carbon atoms (e.g., C₁-C₆), 1-4 carbon atoms (e.g., C₁-C₄), 1-3 carbon atoms (e.g., C₁-C₃), or 2-8 carbon atoms (e.g., C₂-C₈) as used with respect to any chemical group (e.g., alkyl, alkylamino, etc.) referenced herein encompasses and specifically describes 1, 2, 3, 4, 5, 6, 7, 8, 9, 10, 11, and/or 12 carbon atoms, as appropriate, as well as any sub-range thereof (e.g., 1-2 carbon atoms, 1-3 carbon atoms, 1-4 carbon atoms, 1-5 carbon atoms, 1-6 carbon atoms, 1-7 carbon atoms, 1-8 carbon atoms, 1-9 carbon atoms, 1-10 carbon atoms, 1-11 carbon atoms, 1-12 carbon atoms, 2-3 carbon atoms, 2-4 carbon atoms, 2-5 carbon atoms, 2-6 carbon atoms, 2-7 carbon atoms, 2-8 carbon atoms, 2-9 carbon atoms, 2-10 carbon atoms, 2-11 carbon atoms, 2-12 carbon atoms, 3-4 carbon atoms, 3-5 carbon atoms, 3-6 carbon atoms, 3-7 carbon atoms, 3-8 carbon atoms, 3-9 carbon atoms, 3-10 carbon atoms, 3-11 carbon atoms, 3-12 carbon atoms, 4-5 carbon atoms, 4-6 carbon atoms, 4-7 carbon atoms, 4-8 carbon atoms, 4-9 carbon atoms, 4-10 carbon atoms, 4-11 carbon atoms, and/or 4-12 carbon atoms, etc., as appropriate).

In light of the disclosures of the present invention, it will be appreciated that the compounds used in the present invention can be obtained by methods known to those of ordinary skill in the art, for example, by structurally modifying a given compound or by direct synthesis from available precursors using routine synthetic transformations that are well-known in the art. For example, a compound of formula I can be synthesized according to the general approach depicted in Scheme I:

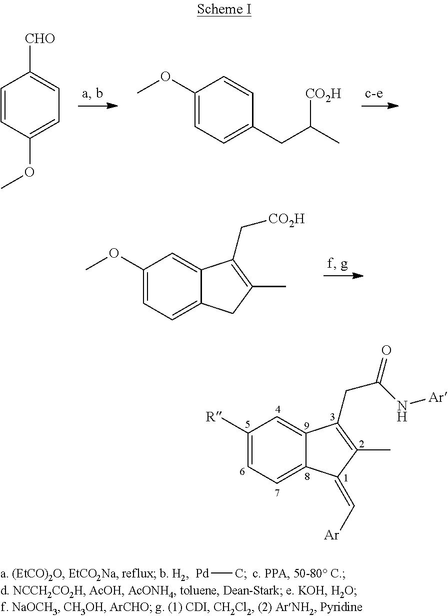

Compounds of formula II can be synthesized according to the general approach depicted in Scheme II:

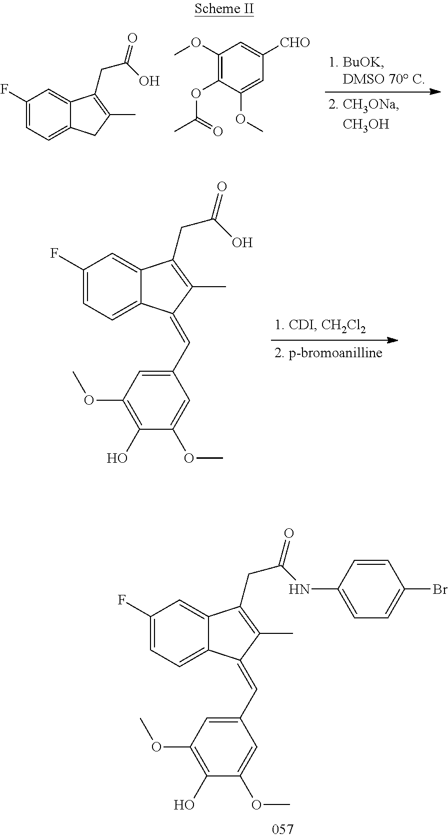

Specific illustrations using these general synthetic approaches are provided in the examples that follow herein. Furthermore, one skilled in the art and knowing the disclosures of the present invention will appreciate that any of compounds of formula I or II can be synthesized using appropriate precursors which can be modified with different substituents as desired to be in the final products, and/or the final product of a synthesis according to Scheme I and II can be modified with different substituents as desired. Placement, removal and/or inter-conversion of desired substituents on precursors, intermediates or penultimate product compounds of formulas I and II can be accomplished by routine methods well-known to those of ordinary skill in the art, as briefly overviewed in the following:

One or more hydroxyl groups, for example, can be converted to the oxo derivative by direct oxidation, which can be accomplished using any known method such as, for example, a Swern oxidation, or by reaction with a metal oxidant, such as a chromium oxide (e.g., chromium trioxide), a manganese oxide (e.g., manganese dioxide or permanganate) or the like. Primary alcohols can be oxidized to aldehydes, for example, via Swern oxidation, or they can be oxidized to carboxylic acids (e.g., —CO₂H), for example by reaction with a metal oxidant. Similarly, the thiols (e.g., —SR, —SH, and the like) can be converted to oxidized sulfur derivatives (e.g., —SO₂R or the like) by reaction with an appropriate oxidant.

One or more hydroxyl groups can be converted to an ester (e.g., —CO₂R), for example, by reaction with an appropriate esterifying agent such as for example, an anhydride (e.g., (R(CO))₂O) or an acid chloride (e.g., R(CO)Cl), or the like. One or more hydroxyl groups can be converted to a sulfonate (e.g., —SO₂R) by reaction with an appropriate sulfonating agent such as, for example, a sulfonyl chloride (e.g., RSO₂Cl), or the like, wherein R is any suitable substituent including, for example, organic substituents described herein. Ester derivatives also can be obtained, for example, by reacting one or more carboxylic acid substituents (e.g., —CO₂H) with an alkylating agent such as, for example, a diazoalkane (e.g., diazomethane) an alkyl or aryl iodide, or the like. One or more amides can be obtained by reaction of one or more carboxylic acids with an amine under appropriate amide-forming conditions which include, for example, activation of a carboxylic acid (e.g., by conversion to an acid chloride or by reaction with a carbodiimide reagent) followed by coupling of the activated species with a suitable amine.

One or more hydroxyl groups can be converted to a halogen using a halogenating agent such as, for example, an N-halosuccinamide such as N-iodosuccinamide, N-bromosuccinamide, N-chlorosuccinamide, or the like, in the presence of a suitable activating agent (e.g., a phosphine, or the like). One or more hydroxyl groups also can be converted to ether by reacting one or more hydroxyls, for example, with an alkylating agent in the presence of a suitable base. Suitable alkylating agents can include, for example, an alkyl or aryl sulfonate, an alkyl or aryl halide, or the like. One or more suitably activated hydroxyls, for example a sulfonate ester, and/or one or more suitably activated halides, can be converted to the corresponding cyano, halo, or amino derivative by displacement with a nucleophile which can include, for example, a thiol, a cyano, a halide ion, or an amine (e.g., H₂NR, wherein R is a desired substituent), or the like.

Amines can be obtained by a variety of methods known in the art, for example, by hydrolysis of one or more amide groups. Amines also can be obtained by reacting one or more suitable oxo groups (e.g., an aldehyde or ketone) with one or more suitable amines under the appropriate conditions, for example, reductive amination conditions, or the like. One or more amines, in turn, can be converted to a number of other useful derivatives such as, for example, amides, sulfonamides, and the like.

Certain chemical modifications of a compound of formula I or II can be introduced as desired to obtain useful new variants with new or modified biological properties such as: new or improved potency and/or selectivity for inhibiting Ras-mediated biological processes, improved efficacy against a disease process such as, but not limited to, tumor cell growth, proliferation, survival, invasion and metastasis, as well as resistance to chemotherapy, other molecularly targeted therapeutics, and radiation, as well as enhanced oral bioavailability, less toxicity in a particular host mammal, more advantageous pharmacokinetics and/or tissue distribution in a given host mammal, and the like. Therefore, the present invention employs methods for obtaining useful new compounds of formula I-II by applying one or more well-known chemical reactions to a given compound to obtain a derivative wherein, for example, one or more phenolic hydroxyl group(s) may instead be replaced by an ester, sulfonate ester or ether group; one or more methyl ether group(s) may instead be replaced by a phenolic hydroxyl group; one or more phenolic hydroxyl group(s) may instead be replaced by an aromatic hydrocarbon substituent; a secondary amine site may instead be replaced by an amide, sulfonamide, tertiary amine, or alkyl quaternary ammonium salt; a tertiary amine site may instead be replaced by a secondary amine; and one or more aromatic hydrogen substituent(s) may instead be replaced by a halogen, nitro, amino, hydroxyl, thiol or cyano substituent.

Depending upon the stoichiometric amount of the particular reactant, a compound of formula I or II can be substituted at one, some, or all of the respective available positions. For example, when such a compound is reacted with a certain amount of CH₃COCl, an acetate substituent can be introduced a one, some, or all of the available positions, which may include, for example ether or amino positions.

Other examples may include, but are not limited to: (1) conversion to ester, sulfonate ester, and ether substituents at one or more phenolic hydroxyl positions in compounds of formula I and II; for instance, for preparation of esters or sulfonate esters a given compound can be reacted with an acid halide (e.g., RCOX or RSO₂X, where X is Cl, Br or I, and R is a C₁-C₆ aliphatic or aromatic radical) in anhydrous pyridine or triethylamine; alternatively, the given compound may be reacted with an acid (RCO₂H or RSO₃H) wherein R is an aliphatic or aromatic radical and dicyclohexylcarbodiimide in triethylamine to prepare the ester or sulfonate ester; for preparation of ethers, the given compound is reacted with an organic halide (e.g., RX or RCH₂X, where X is Cl, Br or I, and R is a C₁-C₆ aliphatic or aromatic radical) in anhydrous acetone with anhydrous potassium carbonate; (2) removal of an ether methyl group(s) to provide a phenolic hydroxyl functionality and/or conversion of that moiety to an ester, sulfonate, or other ether in a compound or derivative of formula I or II: for instance, for hydrolytic cleavage of a methyl ether substituent and conversion to a phenolic hydroxyl moiety, the given compound is reacted with BBr₃ or BX₃.(CH₃)₂S in CH₂Cl₂ (where X is F, Cl or Br); the resulting phenol can be converted to an ester, sulfonate ester or ether as described above; (3) preparation of amide or sulfonamide derivatives at an amine site in a compound of formula I or II: for instance, for preparation of amides or sulfonamide derivatives, the same general procedures described above in (1) apply; in either case (procedure (1) or (3)), an appropriate functional group protection strategy (blocking/deblocking of selected group(s)) may need to be applied; (4) conversion of a secondary amine functionality in a compound of formula I or II to a tertiary amine: for instance, for preparation of a tertiary amine, the given compound is reacted with an aldehyde, and the resulting product is then reduced with NaBH₄; alternatively, for preparation of an alkyl ammonium salt, the given compound is reacted with an alkyl halide (RX, where X is Cl, Br or I, and R is a C₁-C₆ aliphatic radical) in an anhydrous aprotic solvent; (5) conversion of a tertiary amine functionality in a compound of formula I or II to a secondary amine; for instance, for preparation of a secondary amine, the given compound is reacted with cyanogen bromide to give a cyanamide derivative which is then treated with LiAlH₄; (6) conversion of one or more phenolic hydroxyl groups in a given compound of formula I or II to an aromatic hydrogen substituent: for instance, the given compound is converted (after suitable protection of any amine substituent(s) if necessary) to the triflate ester to give the corresponding deoxy compound; (7) substitution of one or more hydrogen substituent(s) on the aryl system(s) on a compound of formula I or II by halogen, nitro, amino, hydroxyl, thiol, or cyano groups: for instance, for preparation of a bromine-substituted derivative, the given compound is reacted with Br₂ in H₂O; for the preparation of other substituted derivatives, the given compound is treated with HNO₃/HOAc to provide a nitro-substituted (—NO₂) derivative; in turn, the nitro-derivative can be reduced to an amino derivative, and the amino derivative is the point of origin of the chloro, iodo, cyano, thiol and hydroxyl substitution via well-known and practiced diazonium substitution reactions. More detailed, specific illustrations of synthesis and derivatization procedures that can be employed to access any desired member of the family of compounds represented by formulas I and II and derivatives thereof, are provided in the examples that follow herein.

It will be appreciated that certain compounds of formula I or II can have one or more asymmetric carbon(s) and thus such compounds are capable of existing as enantiomers or diastereomers. Unless otherwise specified, the present invention includes such enantiomers or diastereomers, including any racemates thereof. If desired, the separate enantiomers or diastereomers can be synthesized from appropriate chiral starting materials, or the racemates can be resolved by conventional procedures, which are well-known to those skilled in the art, such as chiral chromatography, fractional crystallization of diastereomers or diastereomeric salts, and the like. Certain compounds can exist as geometrical isomers, such as, for example, compounds with double-bonded substituents with geometrical isomers Z and E, and the present invention includes all such isomers, including certain isomers, for example the Z isomers, which are preferred. Also, certain compounds may contain substituents wherein there is restricted rotation and/or other geometric isomers are possible. For example, certain oxime substituents may exist in syn or anti configurations. The present invention includes all such configurations, including all possible hindered-rotational isomers, and other geometric isomers.

It will be appreciated by one skilled in the art that the proof or confirmation of the chemical structure of a compound provided by or used in the present invention can be demonstrated using at least one or more well-known and established, convergent methods including, but not limited to, for example: proton and/or carbon NMR spectroscopy, mass spectrometry, x-ray crystallography, chemical degradation, and the like.

One or more compound(s) of formula I or II or pharmaceutically acceptable salt(s) or prodrugs(s) thereof can be included in a composition, e.g., a pharmaceutical composition. In that respect, the present invention further provides a composition that includes an effective amount of at least one compound of formula I or II, which may be in the form of pharmaceutically acceptable salt(s) or prodrug(s) thereof and a pharmaceutically acceptable carrier. The composition of the present invention preferably includes a therapeutically or prophylactically effective amount of at least one Ras-inhibitory compound of formula I or II. The therapeutically or prophylactically effective amount can include an amount that produces a therapeutic or prophylactic response in a patient to whom a compound or composition of the present invention is administered. A therapeutically or prophylactically effective amount can include, for example, a Ras-inhibitory and/or an anticancer effective amount.

The composition of the present invention can further include a therapeutically or prophylactically effective amount of at least one additional compound other than a compound of formula I or II, which may or may not be another Ras-inhibitory compound, and may be an anticancer compound. When the additional compound is a Ras-inhibitory compound other than a compound of formula I or II, it is preferably present in the composition in a Ras-inhibiting amount. When the additional compound is an anticancer compound in general, it is preferably present in the composition in an anticancer effective amount.

The composition of the present invention can be produced by combining one or more compound(s) of formula I or II with an appropriate pharmaceutically acceptable carrier, and can be formulated into a suitable preparation, which may include, for example, preparations in solid, semi-solid, liquid or gaseous forms such as tablets, capsules, powers, granules, ointments, solutions, suppositories, injections, inhalants, and aerosols, and other formulations known in the art for their respective routes of administration. In pharmaceutical dosage forms, a compound of formula I or II can be used alone or in appropriate association, as well as in combination, with other pharmacologically active compounds, including other compounds, e.g., other Ras-inhibitory compounds, as described herein.

Any suitable pharmacologically or physiologically acceptable carrier can be utilized. The following methods and carriers are merely exemplary and are in no way limiting. In the case of oral preparations, a compound of formula I or II can be administered alone or in combination with a therapeutically or prophylactically effective amount of at least one other compound. The active ingredient(s) can be combined, if desired, with appropriate additives to make tablets, powders, granules, capsules or the like.

Suitable additives can include, for example, lactose, mannitol, corn starch or potato starch. Suitable additives also can include binders, for example crystalline cellulose, cellulose derivatives, acacia, or gelatins; disintegrants, for example, corn starch, potato starch or sodium carboxymethylcellulose; or lubricants such as talc or magnesium stearate. If desired, other additives such as, for example, diluents, buffering agents, moistening agents, preservatives, and/or flavoring agents, and the like, can be included in the composition.

The Ras-inhibitory compounds used in accordance with the present invention can be formulated into a preparation for injection or infusion by dissolution, suspension, or emulsification in an aqueous or non-aqueous solvent, such as vegetable oil, synthetic aliphatic acid glycerides, esters of higher aliphatic acid or propylene glycol (if desired, with conventional additives such as solubilizers isotonic agents, suspending agents, emulsifying agents, stabilizers, and preservatives).

The compounds of formula I and II also can be made into an aerosol formulation to be administered by inhalation. Such aerosol formulations can be placed into pressurized acceptable propellants such as dichlorodifluoromethane, propane, nitrogen, and the like.

The compounds can be formulated into suppositories by admixture with a variety of bases such as emulsifying bases or water-soluble bases. The suppository formulations can be administered rectally, and can include vehicles such as cocoa butter, carbowaxes, and polyethylene glycols, which melt at body temperature but are solid at room temperature.

Unit dosage forms for oral or rectal administration such as syrups, elixirs, and suspensions can be provided wherein each dosage unit, e.g., teaspoonful, tablet, or suppository contains a predetermined amount of the composition containing the compound of formula I or II. Similarly, unit dosage forms for injection or intravenous administration can comprise a composition as a solution in sterile water, normal saline, or other pharmaceutically acceptable carrier.

The term “unit dosage form” as used herein refers to physically discrete units suitable as unitary dosages for human and animal subjects, each unit containing a predetermined quantity of at least one compound(s) of formula I or II (alone, or if desired, with another therapeutic or prophylactic agent). The unit dosage can be determined by methods known to those of skill in the art, for example, by calculating the amount of active ingredient sufficient to produce the desired effect in association with a pharmaceutically acceptable carrier. The specifications for the unit dosage forms that can be used in accordance with the present invention depend on the particular effect to be achieved and the particular pharmacodynamics associated with the compound(s) in the individual host.

Pharmaceutically acceptable carriers, for example, vehicles, adjuvants, excipients, or diluents, are accessible to those of skill in the art and are typically available commercially. One skilled in the art can easily determine the appropriate method of administration for the exact formulation of the composition being used. Any necessary adjustments in dose can readily be made by an ordinarily skilled practitioner to address the nature or severity of the condition being treated. Adjustments in dose also can be made on the basis of other factors such as, for example, the individual patient's overall physical health, sex age, prior medical history, and the like.

The compounds of formula I and II can be utilized in a variety of therapeutic and prophylactic (disease preventing) applications, and also in certain non-therapeutic or non-prophylactic applications. It will be appreciated that one or more of these compounds can be used, for example, as a control in diagnostic kits, bioassays, or the like. Preferably the method of the present invention is applied therapeutically or prophylactically, for example, toward treatment or prevention of cancer or toward treatment or prevention of a condition (e.g. an abnormal condition or disease) treatable by the inhibition of Ras-mediated biological process(es). The compounds of formula I and II can be administered alone, or in combination with a therapeutically or prophylactically effective amount of at least one additional compound other than a compound of formula I or II.

Accordingly, the present invention further provides a method of therapeutically or prophylactically treating a condition treatable by the inhibition of one or more Ras-mediated biological processes, which method includes administering to a patient a Ras-inhibiting amount of at least one Ras-inhibitory compound of formula I or II. More particularly, the present invention provides a method of therapeutically or prophylactically treating a condition treatable by the inhibition of one or more Ras-mediated biological processes, which includes administering a Ras-inhibiting effective amount of at least one compound of formula I or II.

A number of conditions can be treated in accordance with the method of the present invention. The compounds of formulas I and II, and their compositions can be used medically to regulate biological phenomena, including but not limited to such Ras-modulated processes as tumor cell growth, proliferation, survival, invasion and metastasis, as well as resistance to chemotherapy, other molecularly targeted therapeutics, and radiation. The compounds of formula I and II are therefore useful in the treatment of diseases and conditions that can be controlled by the inhibition of Ras-mediated cellular functions. Such diseases include, for example, diseases wherein hyperactive Ras (e.g., including mutant Ras) is implicated; such diseases prominently include cancer, among others. Compounds of formula I or II can be expected to have efficacious actions in patients with cancer, especially in patients whose cancers have underlying hyperactive, over-expressed or mutant Ras-mediated pathological processes that are inhibited by a compound(s) of formula I or II. Other aberrant Ras-mediated diseases or conditions that are expected to be treatable or preventable by administration of Ras-inhibiting amounts of compound(s) of formula I or II include for example, neurofibromatosis and Costello syndrome. In the instance of cancer particularly, compound(s) of formula I or II may promote broader sensitivity of cancer to other drugs and/or radiation therapy by inhibiting the ability of cancer cells to develop or express resistance to such drugs and/or radiation therapy making possible the effective chemotherapeutic and/or radiotherapeutic treatment of cancer.

In accordance with an embodiment of the method of the present intervention, it is preferred that a Ras-inhibiting effective amount is used. In that regard, it is preferred that the Ras-inhibiting amount is effective to inhibit one or more conditions selected from the group consisting of tumor cell growth, proliferation, survival, invasion and metastasis, as well as resistance to chemotherapy, other molecularly targeted therapeutics, and radiation.

The method of the present invention further includes administering a Ras-inhibiting effective amount of at least one additional compound other than a compound of formula I or II. In some instances, the method of the present invention can be made more effective by administering one or more other Ras-inhibitory compound(s), along with a compound of formula I or II. One or more Ras-inhibitory compound(s) of formula I or II also can be co-administered in combination with an anticancer agent other than a compound of formula I or II, for example, to cause anticancer chemotherapy-resistant and/or radiation-resistant tumor cells to become chemotherapy-sensitive and/or radiation-sensitive and/or to inhibit de novo the development of cancer cell resistance to the anticancer agent and/or to cancer cell resistance to radiation treatment.

In accordance with an embodiment of the method, the patient is pre-selected by utilizing an assay of said patient's tissue, blood or tumor for an abnormal, mutant or hyperactive ras gene or Ras protein, or an aberrant Ras-mediated biological process.

In accordance with the methods of the present invention, one or more compounds of formula I or II can be administered by any suitable route including, for example, oral or parenteral, including intravenous, subcutaneous, intraarterial, intraperitoneal, ophthalmic, intramuscular, buccal, rectal, vaginal, intraorbital, intracerebral, intracranial, intraspinal, intraventricuclar, intrathecal, intracisternal, intracapsular, intrapulmonary, intranasal, transmucosal, transdermal, or via inhalation. For example, one or more compound(s) of formula I or II can be administered as a solution that is suitable for intravenous injection or infusion, or can be administered as a tablet, a capsule, or the like, in any other suitable composition or formulation as described herein. Accordingly, there is a wide variety of suitable formulations of the pharmaceutical composition of the present invention. The formulations may also be applied topically.

The Ras-“inhibiting-effective amount” as utilized in accordance with an embodiment of the composition and method of the present invention, includes the dose necessary to achieve a Ras-“inhibiting effective level” of the active compound in an individual patient. The Ras-inhibiting-effective amount can be defined, for example, as that amount required to be administered to an individual patient to achieve in said patient a Ras-inhibiting-effective blood or tissue level, and/or intracellular target-inhibiting level of a compound of formula I or II to cause the desired medical treatment.

By way of example and not intending to limit the invention, the dose of the pharmaceutically active agent(s) described herein for methods of preventing or treating a disease or disorder can be, in embodiments, about 0.001 to about 1 mg/kg body weight of the subject being treated per day, for example, about 0.001 mg, 0.002 mg, 0.005 mg, 0.010 mg, 0.015 mg, 0.020 mg, 0.025 mg, 0.050 mg, 0.075 mg, 0.1 mg, 0.15 mg, 0.2 mg, 0.25 mg, 0.5 mg, 0.75 mg, or 1 mg/kg body weight per day. In certain embodiments, the dose of the pharmaceutically active agent(s) described herein can be about 1 to about 1000 mg/kg body weight of the subject being treated per day, for example, about 1 mg, 2 mg, 5 mg, 10 mg, 15 mg, 0.020 mg, 25 mg, 50 mg, 75 mg, 100 mg, 150 mg, 200 mg, 250 mg, 500 mg, 750 mg, or 1000 mg/kg body weight per day.

The terms “treat,” “prevent,” “ameliorate,” and “inhibit,” as well as words stemming therefrom, as used herein, do not necessarily imply 100% or complete treatment, prevention, amelioration, or inhibition. Rather, there are varying degrees of treatment, prevention, amelioration, and inhibition of which one of ordinary skill in the art recognizes as having a potential benefit or therapeutic effect. In this respect, the inventive methods can provide any amount of any level of treatment, prevention, amelioration, or inhibition of the disorder in a mammal. For example, a disorder, including symptoms or conditions thereof, may be reduced by, for example, 100%, 90%, 80%, 70%, 60%, 50%, 40%, 30%, 20%, or 10%. Furthermore, the treatment, prevention, amelioration, or inhibition provided by the inventive method can include treatment, prevention, amelioration, or inhibition of one or more conditions or symptoms of the disorder, e.g., cancer. Also, for purposes herein, “treatment,” “prevention,” “amelioration,” or “inhibition” can encompass delaying the onset of the disorder, or a symptom or condition thereof.

When the effective level is used as the preferred endpoint for dosing, the actual dose and schedule can vary depending, for example, upon inter-individual differences in pharmacokinetics, drug distribution, metabolism, and the like. The effective level also can vary when one or more compound(s) of formula I or II are used in combination with other therapeutic agents, for example, one or more additional anticancer compound(s), or a combination thereof. Moreover, the effective level can vary depending upon the particular disease (e.g., cancer or neurofibromatosis) or biological process (e.g., tumor cell growth, proliferation, survival, invasion and metastasis, as well as resistance to chemotherapy, other molecularly targeted therapeutics, and radiation) for which treatment is desired. Similarly, the effective level can vary depending on whether the treatment is for therapy or prevention of a particular disease such as, for example, cancer.

Compounds of formula I and II can be expected to be broadly efficacious anticancer agents, which will inhibit or destroy human solid tumors, and as well non-solid cancer such as leukemias and certain lymphomas. Solid tumors may include particularly those tumors where ras gene mutations are highly prevalent, such as pancreatic cancer, lung cancer and colon cancer, as well as diverse other solid tumors such as, for example, melanoma, ovarian cancer, renal cancer, prostate cancer, head and neck cancer, endocrine tumors, uterine cancer, breast cancer, sarcomas, gastric cancer, hepatic cancer, esophageal cancer, central nervous system (e.g., brain) cancer, hepatic cancer, germline cancer, and the like.

In a preferred embodiment of the present invention, patients who are most likely to have a favorable response to a Ras-inhibitory compound of formula I or II can be pre-selected, prior to said treatment with said compound, by assaying said patient's blood, tissues or tumor for the presence ras gene mutations and/or abnormal Ras proteins and/or aberrant Ras-mediated biological function(s), using assay procedures (including use of commercially available assay kits) well-known to those of ordinary skill in the art.

Accordingly, the present invention further provides a method of therapeutically or prophylactically treating cancer, which method comprises administering to a patient in need thereof an anticancer effective amount of at least one Ras-inhibitory compound(s) of formula I or II. The anticancer effective amount can be determined, for example, by determining an amount to be administered effective to produce a Ras-inhibiting-effective blood or tissue level and/or intracellular target-inhibiting “effective level” in the subject patient. The effective level can be chosen, for example, as that blood and/or tissue level (e.g., 10⁻¹²-10⁻⁶ M from examples that follow) effective to inhibit the proliferation of tumor cells in a screening assay. Similarly, the effective level can be determined, for example, on the basis of the blood, tissue or tumor level in a patient that corresponds to a concentration of a therapeutic agent that effectively inhibits the growth of a human cancer in any assay that is clinically predictive of anticancer activity. Further, the effective level can be determined, for example based on a concentration at which certain markers of cancer in a patient's blood or tumor tissue (e.g., mutant or hyperactive ras gene(s) and/or Ras protein(s) and/or aberrant Ras-mediated biological pathway(s)) are inhibited by a particular compound that inhibits cancer. Alternatively, the effective level can be determined, for example, based on a concentration effective to slow or stop the growth of a patient's cancer, or cause a patient's cancer to regress or disappear, or render a patient asymptomatic to a particular cancer, or improve a cancer patient's subjective sense of condition. The anticancer effective level can then be used to approximate (e.g., by extrapolation) or even to determine precisely, the level which is required clinically to achieve a Ras-inhibiting-effective blood, tissue, tumor and/or intracellular level to cause the desired medical treatment. It will be appreciated that the determination of the therapeutically effective amount clinically required to effectively inhibit Ras-mediated processes also requires consideration of other variables that can influence the effective level, as discussed herein. When a fixed effective amount is used as a preferred endpoint for dosing, the actual dose and dosing schedule for drug administration can vary for each patient depending upon factors that include, for example, inter-individual differences in pharmacokinetics, drug absorption, drug disposition and tissue distribution, drug metabolism, drug excretion, whether other drugs are used in combination, or other factors described herein that influence the effective level.

One skilled in the art and knowing and understanding the disclosures of the present invention can readily determine the appropriate dose, schedule, or method of administering a particular formulation, in order to achieve the desired effective level in an individual patient. Given the disclosures herein, one skilled in the art also can readily determine and use an appropriate indicator of the effective level of the compound(s) of formula I and II. For example, the effective level can be determined by direct analysis (e.g., analytical chemistry) or by indirect analysis (e.g., with clinical chemistry indicators) of appropriate patient samples (e.g., blood and/or tissues). The effective level also can be determined, for example, if the compound in question has antitumor activity, by direct or indirect observations, such as, for example, observing the shrinkage, slowing or cessation of growth or spreading of a tumor in a cancer patient. There are many references to the art that describe the protocols used in administering and monitoring responses to active compounds in a patient in need thereof. For example, drug-appropriate protocols used in the administration of different types of anticancer agents to patients are described in “Cancer Chemotherapy and Biotherapy: Principles and Practice” eds. Chabner and Longo, Lippincott, Williams and Wilkins (2011), and citations therein.

The present inventive method of therapeutically or prophylactically treating cancer further includes administering an anticancer effective amount of at least one additional compound other than a compound of formula I or II. For example, one or more compound(s) of formula I or II can be co-administered with an anticancer agent, and/or can be co-administered with radiation therapy, in which case the effective level is the level needed to inhibit or reverse the ability of the cancer to develop resistance to the anticancer agent and/or to the radiation therapy, respectively.

Examples of anticancer compounds include reversible DNA binders, DNA alkylators, and DNA strand breakers. Examples of suitable reversible DNA binders include topetecan hydrochloride, irinotecan (CPT11—Camptosar), rubitecan, exatecan, nalidixic acid, TAS-103, etoposide, acridines (e.g., amsacrine, aminocrine), actinomycins (e.g., actinomycin D), anthracyclines (e.g., doxorubicin, daunorubicin), benzophenainse, XR 11576/MLN 576, benzopyridoindoles, Mitoxantrone, AQ4, Etopside, Teniposide, (epipodophyllotoxins), and bisintercalating agents such as triostin A and echinomycin.

Examples of suitable DNA alkylators include sulfur mustard, the nitrogen mustards (e.g., mechlorethamine), chlorambucil, melphalan, ethyleneimines (e.g., triethylenemelamine, carboquone, diaziquone), methyl methanesulfonate, busulfan, CC-1065, duocarmycins (e.g., duocarmycin A, duocarmycin SA), metabolically activated alkylating agents such as nitrosoureas (e.g., carmustine, lomustine, (2-chloroethyl)nitrosoureas), triazine antitumor drugs such as triazenoimidazole (e.g., dacarbazine), mitomycin C, leinamycin, and the like.

Examples of suitable DNA strand breakers include doxorubicin and daunorubicin (which are also reversible DNA binders), other anthracyclines, belomycins, tirapazamine, enediyne antitumor antibiotics such as neocarzinostatin, esperamicins, calicheamicins, dynemicin A, hedarcidin, C-1027, N1999A2, esperamicins, zinostatin, and the like.

Examples of anticancer agents include abarelix, aldesleukin, alemtuzumab, altretamine, amifostine, aminoglutethimide, anastrazole, arsenic trioxide, asparaginase, azacitidine, azathioprine, BCG vaccine, bevacizumab, bexarotene, bicalutamide, bleomycin sulfate, bortezomib, bromocriptine, busulfan, capecitabine, carboplatin, carmustine, cetuximab, chlorambucil, chloroquine phosphate, cladribine, cyclophosphamide, cyclosporine, cytarabine, dacarbazine, dactinomycin, daunorubicin hydrochloride, daunorubicin citrate liposomal, dexrazoxane, docetaxel, doxorubicin hydrochloride, doxorubicin hydrochloride liposomal, epirubicin hydrochloride, estramustine phosphate sodium, etoposide, estretinate, exemestane, floxuridine, fludarabine phosphate, fluorouracil, fluoxymesterone, flutamide, fulvestrant, gefitinib, gemcitabine hydrochloride, gemtuzumab ozogamicin, goserelin acetate, hydroxyurea, idarubicin hydrochloride, ifosfamide, imtinib mesylate, interferon alfa-2a, interferon alfa-2b, irinotecan hydrochloride trihydrate, letrozole, leucovorin calcium, leuprolide acetate, levamisole hydrochloride, lomustine, lymphocyte immune anti-thymocyte globulin (equine), mechlorethamine hydrochloride, medoxyprogestone acetate, melphalan, mercaptopurine, mesna, methotrexate, mitomycin, mitotane, mitoxantrone hydrochloride, nilutamide, oxaliplatin, paclitaxel, pegaspargase, pentostatin, plicamycin, porfimer sodium, procarbazine hydrochloride, streptozocin, tamoxifen citrate, temozolomide, teniposide, testolactone, testosterone propionate, thioguaine, thiotepa, topotecan hydrochloride, tretinoin, uracil mustard, valrubicin, vinblastine sulfate, vincristine sulfate, and vinorelbine.

Suitable forms of radiation therapy include, for example, all forms of radiation therapy approved for commercial use in the United States, and those forms that will become approved in the future, for which radiation resistance thereto can be controlled by a Ras-inhibitory compound of formula I or II.

In accordance with an embodiment of the methods of the present invention, prophylaxis includes inhibition as described herein, e.g., inhibition of the growth or proliferation of cancer cells, or inhibition of aberrant Ras-mediated cellular functions. The inhibition can be, but need not be, 100% inhibition in order to be prophylactically effective, and a clinically desirable benefit can be realized with less than 100% inhibition.

The particular Ras-inhibitory compound(s) of formula I or II used in accordance with the present invention can be selected based upon the potency and/or selectivity for inhibiting Ras-mediated cellular processes, as assessed by in vitro or in vivo assays, and/or based on other pharmacological, toxicological, pharmaceutical or other pertinent considerations that are well-known to those skilled in the art. Routine methods for the specific bioassay, quantitation and comparisons of Ras-inhibitory inhibitory and other biological activities and properties of compounds of formula I and II in various tissues, cells, organelles and other preparations, as well as in vivo testing in animals are well-documented in the literature (e.g., see Teicher and Andrews (eds.), Anticancer Drug Development Guide, Humana (2004), and various authors and chapters therein). More specific illustrations of these and other details pertinent to enablement of the present invention are provided in the examples which follow.

The following examples further illustrate the invention but, of course, should not be construed as in any way limiting its scope.

### Example 1

This example illustrates the synthesis of compounds in accordance with an embodiment of the invention: 054, 055, 056, 057, and 058.

The synthesis of (Z)-2-(5-fluoro-1-(4-hydroxy-3,5-dimethoxybenzylidene)-2-methyl-1H-inden-3-yl)-N-(4-fluorophenyl)acetamide (054) is as follows. A solution of (Z)-2-(1-(3,5-dimethoxy-4-hydroxybenzylidene)-5-fluoro-2-methyl-1H-inden-3-yl)acetic acid (120 mg) and carbonyldiimidazole (100 mg) in 6 mL of anhydrous methylene chloride was stirred at room temperature for 30 min. Then, 4-fluoroaniline (150 mg) in 2.0 mL of pyridine was added and the mixture stirred for 3 h at 50° C. The reaction was quenched by addition of 2 mL of KOH (20%), followed by addition of 50 mL of methylene chloride and 25 mL of water. The organic layer was washed with 2% HCl (aq, 20 mL), then washed with water (20 mL×2), and concentrated. The residue was purified on a silica gel column, recrystallized from acetone/ethyl ether to afford 46 mg of 054 as a yellow solid.

The synthesis of (Z)—N-(4-chlorophenyl)-2-(5-fluoro-1-(4-hydroxy-3,5-dimethoxybenzylidene)-2-methyl-1H-inden-3-yl)acetamide (055) is as follows. A solution of (Z)-2-(1-(3,5-dimethoxy-4-hydroxybenzylidene)-5-fluoro-2-methyl-1H-inden-3-yl)acetic acid (120 mg) and carbonyldiimidazole (100 mg) in 6 mL of anhydrous methylene chloride was stirred at room temperature for 30 min. 4-chloroaniline (150 mg) in 2.0 mL of pyridine was added and the mixture was stirred for 3 h at 50° C., followed by quenching with 2 mL of KOH (20%). 50 mL of methylene chloride and 25 mL of water were added, and the organic layer was washed with 2% HCl (aq, 20 mL), washed with water (20 mL×2), and concentrated. The residue was purified on a silica gel column, recrystallized from acetone/ethyl ether to afford 65 mg of 055 as a yellow solid.

The synthesis of (Z)—N-(3,4-dichlorophenyl)-2-(5-fluoro-1-(4-hydroxy-3,5-dimethoxybenzylidene)-2-methyl-1H-inden-3-yl)acetamide (056) is as follows. A solution of (Z)-2-(1-(3,5-dimethoxy-4-hydroxybenzylidene)-5-fluoro-2-methyl-1H-inden-3-yl)acetic acid (120 mg) and carbonyldiimidazole (100 mg) in 6 mL of anhydrous methylene chloride was stirred at room temperature for 30 min. 3,4-dichloroaniline (150 mg) in 2.0 mL of pyridine was added and the mixture stirred for 3 h at 50° C. The reaction was quenched by addition of 2 mL of KOH (20%), followed by addition of 50 mL of methylene chloride and 25 mL of water. The organic layer was washed with 2% HCl (aq, 20 mL), with water (20 mL×2), and concentrated. The residue was purified on a silica gel column, and recrystallized from acetone/ethyl ether to afford 73 mg of 056 as a yellow solid.

The synthesis of (Z)—N-(4-bromophenyl)-2-(5-fluoro-1-(4-hydroxy-3,5-dimethoxybenzylidene)-2-methyl-1H-inden-3-yl)acetamide (057) is as follows. A solution of (Z)-2-(1-(3,5-dimethoxy-4-hydroxybenzylidene)-5-fluoro-2-methyl-1H-inden-3-yl)acetic acid (120 mg) and carbonyldiimidazole (100 mg) in 6 mL of anhydrous methylene chloride was stirred at room temperature for 30 min, followed by addition of 4-bromoaniline (150 mg) in 2.0 mL of pyridine, and stirred for 3 h at 50° C. The reaction was quenched by addition of 2 mL of KOH (20%), 50 mL of methylene chloride and 25 mL of water were added, the organic layer washed with 2% HCl (aq, 20 mL), then water (20 mL×2), and then concentrated. The residue was purified on a silica gel column, and recrystallized from acetone/ethyl ether to afford 86 mg of 057 as a yellow solid.

The synthesis of (Z)—N-(3-bromophenyl)-2-(5-fluoro-1-(4-hydroxy-3,5-dimethoxybenzylidene)-2-methyl-1H-inden-3-yl)acetamide (058) is as follows. A solution of (Z)-2-(1-(3,5-dimethoxy-4-hydroxybenzylidene)-5-fluoro-2-methyl-1H-inden-3-yl)acetic acid (120 mg) and carbonyldiimidazole (100 mg) in 6 mL of anhydrous methylene chloride was stirred at room temperature for 30 min, 3-bromoaniline (150 mg) in 2.0 mL of pyridine was added, and the mixture stirred for 3 h at 50° C. The reaction was quenched by addition of 2 mL of KOH (20%), 50 mL of methylene chloride and 25 mL of water were added. The organic layer was washed with 2% HCl (aq, 20 mL), washed with water (20 mL×2), then concentrated. The residue was purified on a silica gel column, and recrystallized from acetone/ethyl ether to afford 81 mg of 058 as a yellow solid.

### Example 2

Table 1 provides the ¹H-NMR data confirming structures of exemplary compounds of the invention. All spectra were recorded using DMSO-d⁶ as solvent, at 400 MHz.

TABLE 1
¹H-NMR data of exemplary compounds of the invention

| Cpd. | |
|---|---|
| No. | NMR (δ ppm, DMSO-d⁶) |
| 054 | 10.25(br, 1H, OH), 8.82 (s br, 1H, NH), 7.58 (m, 2H, PhH), 7.48 |
| | (m, 2H, PhH), 7.26 (s, 1H, ═CH), 7.14 (m, 2H, PhH), 7.10 |
| | (m, 1H, indH), 6.84 (s, 2H, Ph′H), 6.77 (m, 1H, indH), 3.74 |
| | (s, 6H, CH₃O), 3.65 (s, 2H, CH2), 2.23 (s, 3H, CH₃) |
| 055 | 10.34 (s br, 1H, OH), 8.82(s br, 1H, NH), 7.64(d, 2H, PhH), 7.58 |
| | (dd, 1H, IndH), 7.36 (d, 2H, PhH), 7.27 (s, 1H, ═CH), 7.21 |
| | (dd, 1H, indH), 6.85(s, 2H, Ph′H), 6.77 (td, 1H, IndH), 3.75 |
| | (s, 6H, OCH₃), 3.66 (s, 2H, CH₂), 2.20 (s, 3H, CH₃) |
| 056 | 10.49 (s br, 1H, OH), 8.83(s br, 1H, NH), 7.89, (s, 1H, PhH), 7.57 |
| | (dd, 1H, indH), 7.57 (d, 1H, PhH), 7.50 (dd, 1H, PhH), 7.26 (s, |
| | 1H, ═CH), 7.25(m, 2H, PhH), 7.11(dd, 1H, indH), 6.86 (s, 2H, |
| | Ph′H), 6.78 (td, 1H, indH), 3.75 (s, 6H, OCH₃), 3.68 (s, 2H, CH₂), |
| | 2.18 (s, 3H, CH₃) |
| 057 | 10.33 (s br, 1H, OH), 8.82(s br, 1H, NH), 7.57 (dd, 1H, indH), |
| | 7.57 (d, 2H, PhH), 7.49 (d, 2H, PhH), 7.26 (s, 1H, ═CH), 7.11 |
| | (dd, 1H, indH), 6.84(s, 2H, Ph′H), 6.77 (td, 1H, IndH), 3.75 (s, |
| | 6H, OCH₃), 3.67 (s, 2H, CH2), 2.18 (s, 3H, CH₃) |
| 058 | 10.37 (s br, 1H, OH), 8.82(s br, 1H, NH), 7.94, (s, 1H, PhH), 7.57 |
| | (dd, 1H, indH), 7.50 (d, 1H, PhH), 7.26 (s, 1H, ═CH), 7.25(m, |
| | 2H, PhH), 7.11(dd, 1H, indH), 6.85 (s, 2H, Ph′H), 6.76 (td, 1H, |
| | indH), 3.75 (s, 6H, OCH₃), 3.66(s, 2H, CH₂), 2.20 (s, 3H, CH₃) |

### Example 3

This example illustrates cell growth assays that can be employed in the present invention. Cells can include A-549, HT-29, MDA-MB-231, Colo-205, Caco2, HCT-116, SW-480, and DLD-1 human cancer cells obtained from the American Type Culture Collection (ATCC). Human tumor cells are cultured using standard methods in RPMI-1640 growth medium supplemented with 5% fetal bovine serum (FBS). Normal rat kidney (NRK) and Ki-Ras transformed NRK cells (K-NRK) are obtained from ATCC, and were cultured according to supplier recommendations. CellTiter-Glo ATP cell growth assay reagents are obtained from Promega and used according to the manufacturer's protocol. Inhibitors of EGFR, Raf, and MEK are obtained from Selleck Chemicals. Cells are plated at a density of 5,000 cells per well in 96-well microplates or 1,250 cells per well in 384-well plates, and allowed to attach for at least 4 h. Test compounds are dissolved in dimethyl sulfoxide (DMSO), and this working stock is further diluted in growth medium for addition to cell cultures. Serial dilutions of the test compound are prepared in growth medium containing an equal amount of DMSO not exceeding 0.2% final concentration. Each compound concentration is tested in at least 3 separate samples per cell line. At the end of a 3-day treatment period, growth inhibition is analyzed using a bioluminescent assay of ATP concentration (Promega CellTiter-Glo) according to the manufacturer's protocol. The resulting luminescence is measured using the luminescence cartridge of the Molecular Devices Spectramax Paradigm microplate reader. The relative growth inhibition for each sample is determined by comparison with the values obtained for vehicle treated control samples. Growth inhibition values are plotted with the GraphPad Prism5 software using the 4-parameter logistic fit to obtain IC₅₀ values, which corresponds to the growth inhibitory potency of the compound.

### Example 4

This example illustrates a Ras binding domain assay that can be used in an embodiment of the present invention to measure Ras activation status. The activation state of Ras in cell lines is assayed using the Active Ras Pull-Down and Detection Kit (Thermo Scientific). Cell lines are cultured as described above. Cells are disrupted with non-ionic detergent, and the active (GTP-bound) Ras is isolated by its high affinity for Raf via precipitate with sepharose-bound GST-Raf fusion protein. The precipitated active Ras is then subjected to polyacrylamide gel electrophoresis (PAGE) and transferred to nitrocellulose membrane (western blot). Detection is achieved using the anti-Ras mouse primary antibody and anti-mouse-horseradish peroxidase conjugated secondary antibody. Paired samples of whole cell lysate are analyzed by western blot for expression level of total Ras protein as well as a gel loading control, glyceraldehyde 3-phosphate dehydrogenase (GAPDH). Digital enhanced chemiluminescence imaging of the resulting western blots is performed using a Syngene G:Box. The intensity of Ras bands from each cell line and the corresponding GAPDH bands are quantitated using NIH ImageJ, and expressed as “relative Ras activation.”

### Example 5

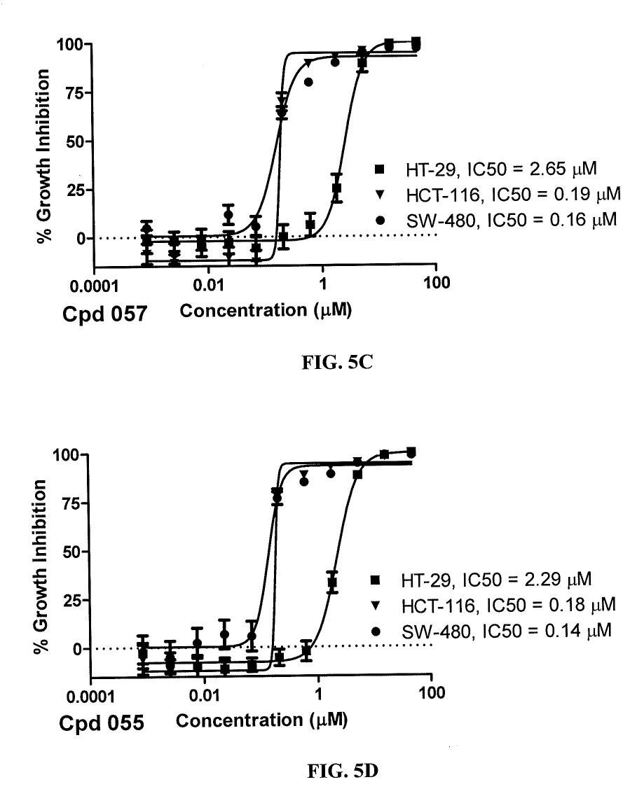

This example illustrates the levels of Ras activation in different colorectal cancer cells. In particular, a panel of human colorectal cancer cell lines was selected to further describe the selectivity of the compounds for cells with activated Ras. Three of the cell lines in the panel have been reported to harbor ras mutations: HCT-116, DLD-1, and SW-480 (Stoneman and Morris, Clin Mol Pathol., 48, M326-332(1995)). Three of the cell lines were reported to express wild type Ras: HT-29, Caco-2, and Colo-205 (Stoneman and Morris, supra; Shirasawa et al., Science, 260, 85-88 (1993)). The activation state of Ras in the cell lines was assayed using the Active Ras Pull-Down and Detection Kit. The ratio of the intensity of the Ras to GAPDH bands was expressed as relative Ras activation, which is presented above each lane in FIG. 4. This experiment demonstrated that the level of Ras activation in HCT-116>DLD-1≈SW-480>Caco2>HT-29>Colo205.

### Example 6

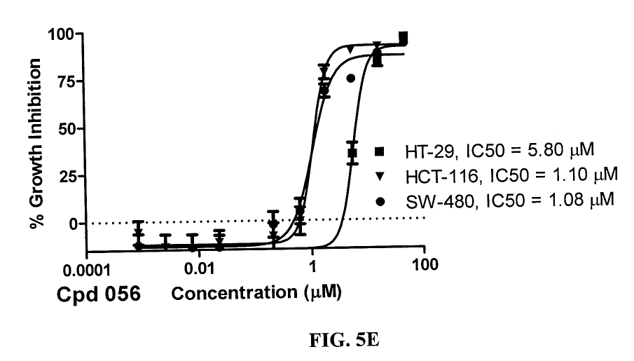

This example illustrates the use of well-established human colon tumor cell lines with widely divergent Ras activation status to determine selectivity values for exemplary compounds of the present invention. Cell lines thus employed in this example were HCT-116, a highly Ras-driven line expressing mutant Ras, SW-480, another strongly Ras-driven line expressing mutant Ras, and HT-29, a non-Ras-driven line expressing wild-type, non-mutated Ras. Cells were plated at 5000 cells/well in 96-well plates, and viable cell numbers were measured using the Cell Titer Glo ATP luminescence assay (Promega). FIGS. 5A-5F show results of these studies for exemplary compounds 054, 058, 057, 055, and 056, respectively. The calculated HT-29/HCT-116 selectivity values for the first five of the aforementioned compounds were 14, 13, 18, 11 and 5, respectively. The calculated HT-29/SW-480 selectivity values for the aforementioned compounds were 16, 16, 19, 13 and 5, respectively. These selectivity values are the ratios of each compound's potency (IC₅₀) to inhibit the growth of cells lacking activated Ras ((HCT-29) relative to that of cells with activated Ras (HCT-116 or SW-480), demonstrating selectivity for the Ras-mutant-containing cells.

This example furthermore shows that prior art studies (e.g., U.S. Pat. Nos. 6,063,818 and 6,121,321) employing individual cell lines such as HT-29 or SW-480 could not have revealed compounds with Ras-selective activity useful for Ras-directed medical treatments or preventions. Employment of a cell line having normal, non-mutant Ras (e.g., HT-29) concurrently with at least one or more cell line(s) having hyperactive or mutant Ras (e.g., HCT-116 and/or SW-480) in comparative assays of tumor cell growth inhibition are required to demonstrate Ras-selectivity, and to enable selection of a Ras-inhibitory compound necessary for the novel methods disclosed in the present invention.

### Example 7

This example illustrates non-selective growth inhibition with known Ras pathway inhibitors which are not compounds of the present invention. In particular, the growth inhibitory activity of commercially available compounds which are active in the Ras signal transduction pathway were tested in the same panel of cell lines using the CellTiter-Glo assay. Cells were seeded in 384-well plates and allowed to attach. Ten-fold serial dilutions of compounds were tested. Each compound concentration was tested in at least 3 separate samples per cell line. As indicated in Table 2 below, the potency of the EGF receptor inhibitor compounds ranged from 4 μM to >20 μM, with no pattern of selectivity with regard to Ras activation. Likewise, the C-Raf inhibitor, GW5074, did not show selectivity for cell lines expressing activated Ras. The B-Raf inhibitors tested were generally active in the low micromolar range, but were significantly more potent in Colo-205 cells, which have the lowest level of active Ras. The MEK inhibitor, Selumetinib was also most potent against COLO-205 and HT-29 cell lines showing, if anything, a “reverse” selectivity toward inactive Ras compared with the compounds of this invention.

TABLE 2
Non Ras-selective growth inhibition with known Ras pathway inhibitors.

| | | HCT-116 | DLD-1 | SW-480 | Caco-2 | HT-29 | COLO-205 |
|---|---|---|---|---|---|---|---|
| | | IC₅₀ | IC₅₀ | IC₅₀ | IC₅₀ | IC₅₀ | IC₅₀ |
| Compound | Target | (nM) | (nM) | (nM) | (nM) | (nM) | (nM) |
| Gefitinib | EGFR | 12,600 | 7,550 | 8,630 | 8,920 | 6,370 | 8,200 |
| GW5074 | C-Raf | 15,800 | 7,750 | 8,070 | >20,000 | 19,100 | 7,100 |
| GDC0879 | B-Raf | 8,140 | 7,640 | >20,000 | >20,000 | 23,600 | 75.8 |
| Vemurafenib | B-Raf | 5,990 | 5,620 | 6,860 | 5,200 | 5,100 | 151 |
| Selumetinib | MEK | 4,110 | 20,400 | >20,000 | >20,000 | 854 | 4.60 |

Together, these data demonstrate the activated Ras selective growth inhibitory activity of the compounds of this invention. This is in contrast to the current clinically used inhibitors of proteins within the Ras signaling cascade that exhibit no selectivity or selectivity for cells lacking activated Ras.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A compound of formula I: 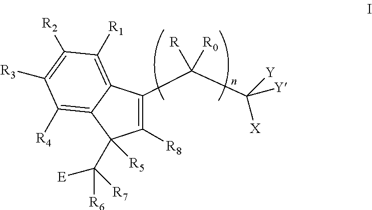
wherein:
R and R₀ are, independently, hydrogen or hydroxyl; n is 0, 1 or 2;
three of R₁, R₂, R₃, and R₄ are hydrogens, and one is halogen, alkyl, or alkoxy, or two of R₁, R₂, R₃, and R₄ are hydrogens and two are alkoxy;
R₅ and R₆ together form a carbon-carbon bond; R₇ is hydrogen; R₈ is alkyl;
Y and Y′ together is oxygen;
X is NR′R″, where R′ is aryl substituted with one or more of halogen, alkyl, haloalkyl, cyano, cyanoalkyl, nitro, hydroxyl, alkoxy, formyloxy, hydroxyalkyl, aldehydo, amino, alkylamino, dialkylamino, aminoalkyl, alkylaminoalkyl, mercapto, and alkylmercapto, azido, and substituted or unsubstituted groups selected from alkylsulfonyl, alkylsulfinyl, alkylsulfinyloxy, alkylsulfonyloxy, alkylcarbonyloxy, carbamate, carbamido, alkoxycarbonyl, alkylaminocarbonyl, aminocarbonyl, and sulfonamido, or any two substituted positions may be occupied by an alkylenedioxy group; and R″ is hydrogen; and
E is a substituted aryl; or
a pharmaceutically acceptable salt thereof or a prodrug thereof, or a corresponding Z or E-isomer.

2. The compound of claim 1, pharmaceutically acceptable salt, or a prodrug thereof, or the corresponding Z or E-isomer, wherein E is an aryl substituted with one or more of halogen, alkyl, haloalkyl, cyano, cyanoalkyl, nitro, hydroxyl, alkoxy, formyloxy, hydroxyalkyl, aldehydo, amino, alkylamino, dialkylamino, aminoalkyl, alkylaminoalkyl, mercapto, alkylmercapto, azido, and substituted or unsubstituted groups selected from alkylsulfonyl, alkylsulfinyl, alkylsulfinyloxy, alkylsulfonyloxy, alkylcarbonyloxy, carbamate, carbamido, alkoxycarbonyl, alkylaminocarbonyl, aminocarbonyl, and sulfonamido or any two substituted positions may be occupied by an alkylenedioxy group.

3. The compound of claim 1 or 2, which has the formula (II): 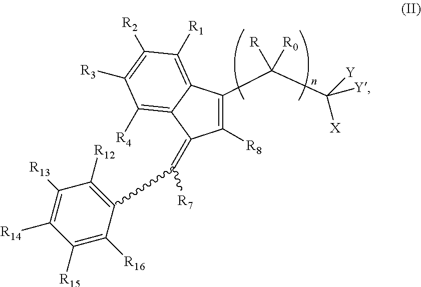
wherein:
R and R₀ are, independently, hydrogen or hydroxyl; n is 0, 1 or 2;
Y and Y′ together is oxygen;
three of R₁, R₂, R₃, and R₄ are hydrogens, and one is halogen, alkyl, or alkoxy, or two of R₁, R₂, R₃, and R₄ are hydrogens and two are alkoxy;
R₇ is hydrogen and R₈ is alkyl;
at least one of R₁₂, R₁₃, R₁₄, R₁₅, and R₁₆ is independently selected from halogen, alkyl, haloalkyl, cyano, cyanoalkyl, nitro, hydroxyl, alkoxy, formyloxy, hydroxyalkyl, aldehydo, amino, alkylamino, aminoalkyl, alkylaminoalkyl, dialkylamino, mercapto, alkylmercapto, azido, and substituted or unsubstituted groups selected from alkylsulfonyl, alkylsulfinyl, alkylsulfinyloxy, alkylsulfonyloxy, alkylcarbonyloxy, carbamate, carbamido, alkoxycarbonyl, alkylaminocarbonyl, aminocarbonyl, and sulfonamido, or any two of R₁₂, R₁₃, R₁₄, R₁₅ and R₁₆ form an alkylenedioxy group; and
X is NR′R″, where R″ is hydrogen; R′ is aryl substituted with one or more of halogen, alkyl, haloalkyl, cyano, cyanoalkyl, nitro, hydroxyl, alkoxy, formyloxy, hydroxyalkyl, aldehydo, amino, alkylamino, dialkylamino, aminoalkyl, alkylaminoalkyl, mercapto, and alkylmercapto, azido, and substituted or unsubstituted groups selected from alkylsulfonyl, alkylsulfinyl, alkylsulfinyloxy, alkylsulfonyloxy, alkylcarbonyloxy, carbamate, carbamido, alkoxycarbonyl, alkylaminocarbonyl, aminocarbonyl, and sulfonamido, or any two substituted positions may be occupied by an alkylenedioxy group;
or a pharmaceutically acceptable salt, or a prodrug thereof, or a corresponding Z or E-isomer.

4. The compound of claim 3, pharmaceutically acceptable salt, or a prodrug thereof, or a corresponding Z or E-isomer, wherein R′ is phenyl or biphenyl substituted with one or more of halogen, alkyl, haloalkyl, cyano, cyanoalkyl, nitro, hydroxyl, alkoxy, formyloxy, hydroxyalkyl, aldehydo, amino, alkylamino, dialkylamino, aminoalkyl, alkylaminoalkyl, mercapto, and alkylmercapto, azido, and substituted or unsubstituted groups selected from alkylsulfonyl, alkylsulfinyl, alkylsulfinyloxy, alkylsulfonyloxy, alkylcarbonyloxy, carbamate, carbamido, alkoxycarbonyl, alkylaminocarbonyl, aminocarbonyl, and sulfonamido, or any two substituted positions may be occupied by an alkylenedioxy group; and at least one of R₁₂, R₁₃, R₁₄, R₁₅ and R₁₆ is independently selected from halogen, alkyl, haloalkyl, cycno, cyanoalkyl, nitro, hydroxyl, alkoxy, formyloxy, hydroxyalkyl, aldehydo, amino, alkylamino, aminoalkyl, alkylaminoalkyl, dialkylamino, mercapto, alkylmercapto, azido, and substituted or unsubstituted groups selected from alkylsulfonyl, alkylsulfinyl, alkylsulfinyloxy, alkylsulfonyloxy, alkylcarbonyloxy, carbamate, carbamido, alkoxycarbonyl, alkylaminocarbonyl, aminocarbonyl, and sulfonamido, or any two of R₁₂, R₁₃, R₁₄, R₁₅ and R₁₆ form an alkylenedioxy group.

5. The compound of claim 3 or 4, pharmaceutically acceptable salt, or a prodrug thereof, or a corresponding Z or E-isomer, wherein R′ is phenyl substituted with one or more of halogen, alkyl, trifluoromethyl, and alkoxy; and one or more of R₁₂, R₁₃, R₁₄, R₁₅ and R₁₆ is independently selected from halogen, alkyl, formyloxy, alkylcarbonyloxy, substituted or unsubstituted groups selected from alkylsulfonyl, alkylsulfinyl, alkylsulfinyloxy, alkylsulfonyloxy, carbamate, carbamido, alkoxycarbonyl, alkylaminocarbonyl, aminocarbonyl, and sulfonamido.

6. The compound of claim 1 or 2, which is selected from:
(Z)-2-(1-(4-hydroxy-3,5-dimethoxybenzylidene)-5-methoxy-2-methyl-1H-inden-3-yl)-N-(m-tolyl)acetamide (036),
(Z)-2-(5-fluoro-1-(4-hydroxy-3,5-dimethoxybenzylidene)-2-methyl-1H-inden-3-yl)-N-(m-tolyl)acetamide (037),
(Z)—N-(3,4-dimethylphenyl)-2-(1-(4-hydroxy-3,5-dimethoxybenzylidene)-5-methoxy-2-methyl-1H-inden-3-yl)acetamide (038),
(Z)—N-(3,4-dimethylphenyl)-2-(5-fluoro-1-(4-hydroxy-3,5-dimethoxybenzylidene)-2-methyl-1H-inden-3-yl)acetamide (039),
(Z)-2-(1-(4-hydroxy-3,5-dimethoxybenzylidene)-5-methoxy-2-methyl-1H-inden-3-yl)-N-(p-tolyl)acetamide (040),
(Z)-2-(5-fluoro-1-(4-hydroxy-3,5-dimethoxybenzylidene)-2-methyl-1H-inden-3-yl)-N-(p-tolyl)acetamide (041),
(Z)-2-(1-(4-hydroxy-3,5-dimethoxybenzylidene)-5-methoxy-2-methyl-1H-inden-3-yl)-N-(o-tolyl)acetamide (042),
(Z)-2-(5-fluoro-1-(4-hydroxy-3,5-dimethoxybenzylidene)-2-methyl-1H-inden-3-yl)-N-(o-tolyl)acetamide (043),
(Z)-2-(1-(4-hydroxy-3-methoxybenzylidene)-5-methoxy-2-methyl-1H-inden-3-yl)-N-(m-tolyl)acetamide (046),
(Z)-2-(5-fluoro-1-(4-hydroxy-3-methoxybenzylidene)-2-methyl-1H-inden-3-yl)-N-(m-tolyl)acetamide (047),
(Z)-2-(1-(4-hydroxy-3-methoxybenzylidene)-5-methoxy-2-methyl-1H-inden-3-yl)-N-(3-methoxyphenyl)acetamide (048),
(Z)-2-(5-fluoro-1-(4-hydroxy-3-methoxybenzylidene)-2-methyl-1H-inden-3-yl)-N-(3-methoxyphenyl)acetamide (049),
(Z)-2-(5-fluoro-1-(4-hydroxy-3,5-dimethoxybenzylidene)-2-methyl-1H-inden-3-yl)-N-(7-methylnaphthalen-2-yl)acetamide (050),
(Z)-4-((5-fluoro-2-methyl-3-(2-oxo-2-(m-tolylamino)ethyl)-1H-inden-1-ylidene)methyl)-2,6-dimethoxyphenyl ethylcarbamate (051),
(Z)-4-((5-fluoro-2-methyl-3-(2-oxo-2-(m-tolylamino)ethyl)-1H-inden-1-ylidene)methyl)-2,6-dimethoxyphenyl methanesulfonate (052),
(Z)-2-(5-fluoro-1-(4-acetoxy-3,5-dimethoxybenzylidene)-2-methyl-1H-inden-3-yl)-N-(m-tolyl)acetamide (053), and
(Z)—N-(4-(tert-butyl)phenyl)-2-(5-fluoro-1-(4-hydroxy-3,5-dimethoxybenzylidene)-2-methyl-1H-inden-3-yl)acetamide (059), or
a pharmaceutically acceptable salt or prodrug thereof, or the corresponding E-isomer thereof.

7. A pharmaceutical composition comprising a compound of any one of claims 1-6, a pharmaceutically acceptable salt or prodrug thereof, or the corresponding Z or E-isomer thereof, and a pharmaceutically acceptable carrier.

8. The pharmaceutical composition of claim 7, further including at least one additional therapeutic agent other than a compound of formula I, a pharmaceutically acceptable salt or prodrug thereof, or the corresponding Z or E-isomer thereof.

9. A method of inhibiting a human or nonhuman mammalian Ras-mediated biological process, which method comprising administering in vivo or in vitro a Ras-inhibitory amount of at least one compound of any one of claims 1-6, a pharmaceutically acceptable salt or prodrug thereof, wherein the compound or pharmaceutically acceptable salt or prodrug thereof or Z or E-isomer is administered alone or in combination with at least one additional therapeutic agent other than said compound or pharmaceutically acceptable salt or prodrug thereof or Z or E-isomer.

10. A method of therapeutically or prophylactically treating a human or nonhuman mammalian patient with a disease or condition treatable by inhibition of one or more Ras-mediated biological process, which method comprising administering to a patient in need thereof a therapeutically or prophylactically effective amount of at least one ras-inhibitory compound of any one of claims 1-6, a pharmaceutically acceptable salt or prodrug thereof, wherein the compound or pharmaceutically acceptable salt or prodrug thereof or Z or E-isomer is administered alone or in combination with at least one additional therapeutic agent other than said compound or pharmaceutically acceptable salt or prodrug thereof or Z or E-isomer.

11. A method of inhibiting a human or nonhuman mammalian Ras-mediated biological process, which method comprising administering in vivo or in vitro a Ras-inhibitory amount of at least one compound selected from:
(Z)-2-(1-(4-hydroxy-3,5-dimethoxybenzylidene)-5-methoxy-2-methyl-1H-inden-3-yl)-N-(m-tolyl)acetamide (036),
(Z)-2-(5-fluoro-1-(4-hydroxy-3,5-dimethoxybenzylidene)-2-methyl-1H-inden-3-yl)-N-(m-tolyl)acetamide (037),
(Z)—N-(3,4-dimethylphenyl)-2-(1-(4-hydroxy-3,5-dimethoxybenzylidene)-5-methoxy-2-methyl-1H-inden-3-yl)acetamide (038),
(Z)—N-(3,4-dimethylphenyl)-2-(5-fluoro-1-(4-hydroxy-3,5-dimethoxybenzylidene)-2-methyl-1H-inden-3-yl)acetamide (039),
(Z)-2-(1-(4-hydroxy-3,5-dimethoxybenzylidene)-5-methoxy-2-methyl-1H-inden-3-yl)-N-(p-tolyl)acetamide (040),
(Z)-2-(5-fluoro-1-(4-hydroxy-3,5-dimethoxybenzylidene)-2-methyl-1H-inden-3-yl)-N-(p-tolyl)acetamide (041),
(Z)-2-(1-(4-hydroxy-3,5-dimethoxybenzylidene)-5-methoxy-2-methyl-1H-inden-3-yl)-N-(o-tolyl)acetamide (042),
(Z)-2-(5-fluoro-1-(4-hydroxy-3,5-dimethoxybenzylidene)-2-methyl-1H-inden-3-yl)-N-(o-tolyl)acetamide (043),
(Z)-2-(1-(4-hydroxy-3,5-dimethoxybenzylidene)-5-methoxy-2-methyl-1H-inden-3-yl)-N-(3-methoxyphenyl)acetamide (044),
(Z)-2-(5-fluoro-1-(4-hydroxy-3,5-dimethoxybenzylidene)-2-methyl-1H-inden-3-yl)-N-(3-methoxyphenyl)acetamide (045),
(Z)-2-(1-(4-hydroxy-3-methoxybenzylidene)-5-methoxy-2-methyl-1H-inden-3-yl)-N-(m-tolyl)acetamide (046),
(Z)-2-(5-fluoro-1-(4-hydroxy-3-methoxybenzylidene)-2-methyl-1H-inden-3-yl)-N-(m-tolyl)acetamide (047),
(Z)-2-(1-(4-hydroxy-3-methoxybenzylidene)-5-methoxy-2-methyl-1H-inden-3-yl)-N-(3-methoxyphenyl)acetamide (048),
(Z)-2-(5-fluoro-1-(4-hydroxy-3-methoxybenzylidene)-2-methyl-1H-inden-3-yl)-N-(3-methoxyphenyl)acetamide (049),
(Z)-2-(5-fluoro-1-(4-hydroxy-3,5-dimethoxybenzylidene)-2-methyl-1H-inden-3-yl)-N-(7-methylnaphthalen-2-yl)acetamide (050),
(Z)-4-((5-fluoro-2-methyl-3-(2-oxo-2-(m-tolylamino)ethyl)-1H-inden-1-ylidene)methyl)-2,6-dimethoxyphenyl ethylcarbamate (051),
(Z)-4-((5-fluoro-2-methyl-3-(2-oxo-2-(m-tolylamino)ethyl)-1H-inden-1-ylidene)methyl)-2,6-dimethoxyphenyl methanesulfonate (052),
(Z)-2-(5-fluoro-1-(4-acetoxy-3,5-dimethoxybenzylidene)-2-methyl-1H-inden-3-yl)-N-(m-tolyl)acetamide (053),
(Z)-2-(5-fluoro-1-(4-hydroxy-3,5-dimethoxybenzylidene)-2-methyl-1H-inden-3-yl)-N-(4-fluorophenyl)acetamide (054),
(Z)—N-(4-chlorophenyl)-2-(5-fluoro-1-(4-hydroxy-3,5-dimethoxybenzylidene)-2-methyl-1H-inden-3-yl)acetamide (055),
(Z)—N-(3,4-dichlorophenyl)-2-(5-fluoro-1-(4-hydroxy-3,5-dimethoxybenzylidene)-2-methyl-1H-inden-3-yl)acetamide (056),
(Z)—N-(4-bromophenyl)-2-(5-fluoro-1-(4-hydroxy-3,5-dimethoxybenzylidene)-2-methyl-1H-inden-3-yl)acetamide (057),
(Z)—N-(3-bromophenyl)-2-(5-fluoro-1-(4-hydroxy-3,5-dimethoxybenzylidene)-2-methyl-1H-inden-3-yl)acetamide (058), and
(Z)—N-(4-(tert-butyl)phenyl)-2-(5-fluoro-1-(4-hydroxy-3,5-dimethoxybenzylidene)-2-methyl-1H-inden-3-yl)acetamide (059), or
a pharmaceutically acceptable salt or prodrug thereof, or the corresponding E-isomer thereof,
wherein said compound, pharmaceutically acceptable salt or prodrug thereof or E-isomer thereof, is administered alone or in combination with at least one additional therapeutic agent other than said compound, pharmaceutically acceptable salt, prodrug, or E-isomer thereof.

12. A method of therapeutically or prophylactically treating a human or nonhuman mammalian patient with a disease or condition treatable by inhibition of one or more Ras-mediated biological process, which method comprising administering to a patient in need thereof a therapeutically or prophylactically effective amount of at least one ras-inhibitory compound selected from:
(Z)-2-(1-(4-hydroxy-3,5-dimethoxybenzylidene)-5-methoxy-2-methyl-1H-inden-3-yl)-N-(m-tolyl)acetamide (036),
(Z)-2-(5-fluoro-1-(4-hydroxy-3,5-dimethoxybenzylidene)-2-methyl-1H-inden-3-yl)-N-(m-tolyl)acetamide (037),
(Z)—N-(3,4-dimethylphenyl)-2-(1-(4-hydroxy-3,5-dimethoxybenzylidene)-5-methoxy-2-methyl-1H-inden-3-yl)acetamide (038),
(Z)—N-(3,4-dimethylphenyl)-2-(5-fluoro-1-(4-hydroxy-3,5-dimethoxybenzylidene)-2-methyl-1H-inden-3-yl)acetamide (039),
(Z)-2-(1-(4-hydroxy-3,5-dimethoxybenzylidene)-5-methoxy-2-methyl-1H-inden-3-yl)-N-(p-tolyl)acetamide (040),
(Z)-2-(5-fluoro-1-(4-hydroxy-3,5-dimethoxybenzylidene)-2-methyl-1H-inden-3-yl)-N-(p-tolyl)acetamide (041),
(Z)-2-(1-(4-hydroxy-3,5-dimethoxybenzylidene)-5-methoxy-2-methyl-1H-inden-3-yl)-N-(o-tolyl)acetamide (042),
(Z)-2-(5-fluoro-1-(4-hydroxy-3,5-dimethoxybenzylidene)-2-methyl-1H-inden-3-yl)-N-(o-tolyl)acetamide (043),
(Z)-2-(1-(4-hydroxy-3,5-dimethoxybenzylidene)-5-methoxy-2-methyl-1H-inden-3-yl)-N-(3-methoxyphenyl)acetamide (044),
(Z)-2-(5-fluoro-1-(4-hydroxy-3,5-dimethoxybenzylidene)-2-methyl-1H-inden-3-yl)-N-(3-methoxyphenyl)acetamide (045),
(Z)-2-(1-(4-hydroxy-3-methoxybenzylidene)-5-methoxy-2-methyl-1H-inden-3-yl)-N-(m-tolyl)acetamide (046),
(Z)-2-(5-fluoro-1-(4-hydroxy-3-methoxybenzylidene)-2-methyl-1H-inden-3-yl)-N-(m-tolyl)acetamide (047),
(Z)-2-(1-(4-hydroxy-3-methoxybenzylidene)-5-methoxy-2-methyl-1H-inden-3-yl)-N-(3-methoxyphenyl)acetamide (048),
(Z)-2-(5-fluoro-1-(4-hydroxy-3-methoxybenzylidene)-2-methyl-1H-inden-3-yl)-N-(3-methoxyphenyl)acetamide (049),
(Z)-2-(5-fluoro-1-(4-hydroxy-3,5-dimethoxybenzylidene)-2-methyl-1H-inden-3-yl)-N-(7-methylnaphthalen-2-yl)acetamide (050),
(Z)-4-((5-fluoro-2-methyl-3-(2-oxo-2-(m-tolylamino)ethyl)-1H-inden-1-ylidene)methyl)-2,6-dimethoxyphenyl ethylcarbamate (051),
(Z)-4-((5-fluoro-2-methyl-3-(2-oxo-2-(m-tolylamino)ethyl)-1H-inden-1-ylidene)methyl)-2,6-dimethoxyphenyl methanesulfonate (052),
(Z)-2-(5-fluoro-1-(4-acetoxy-3,5-dimethoxybenzylidene)-2-methyl-1H-inden-3-yl)-N-(m-tolyl)acetamide (053),
(Z)-2-(5-fluoro-1-(4-hydroxy-3,5-dimethoxybenzylidene)-2-methyl-1H-inden-3-yl)-N-(4-fluorophenyl)acetamide (054),
(Z)—N-(4-chlorophenyl)-2-(5-fluoro-1-(4-hydroxy-3,5-dimethoxybenzylidene)-2-methyl-1H-inden-3-yl)acetamide (055),
(Z)—N-(3,4-dichlorophenyl)-2-(5-fluoro-1-(4-hydroxy-3,5-dimethoxybenzylidene)-2-methyl-1H-inden-3-yl)acetamide (056),
(Z)—N-(4-bromophenyl)-2-(5-fluoro-1-(4-hydroxy-3,5-dimethoxybenzylidene)-2-methyl-1H-inden-3-yl)acetamide (057),
(Z)—N-(3-bromophenyl)-2-(5-fluoro-1-(4-hydroxy-3,5-dimethoxybenzylidene)-2-methyl-1H-inden-3-yl)acetamide (058), and
(Z)—N-(4-(tert-butyl)phenyl)-2-(5-fluoro-1-(4-hydroxy-3,5-dimethoxybenzylidene)-2-methyl-1H-inden-3-yl)acetamide (059), or
a pharmaceutically acceptable salt or prodrug thereof, or the corresponding E-isomer thereof,
wherein said compound, pharmaceutically acceptable salt or prodrug thereof, or E-isomer thereof, is administered alone or in combination with at least one additional therapeutic agent other than said compound, pharmaceutically acceptable salt, prodrug, or E-isomer thereof.

13. The method of any one of claims 9-12, wherein the Ras-mediated biological process is selected from growth, proliferation, survival, metastasis, drug resistance and radiation resistance of a tumor cell.

14. The method of claim 13, wherein the Ras-mediated biological process is cancer.

15. The method of claim 13 or 14, wherein the cancer is selected from pancreatic cancer, lung cancer, colorectal cancer, melanoma, ovarian cancer, renal cancer, prostate cancer, head and neck cancer, endocrine cancer, uterine cancer, breast cancer, sarcoma cancer, gastric cancer, hepatic cancer, esophageal cancer, central nervous system cancer, brain cancer, hepatic cancer, germline cancer, lymphoma, and leukemia.

16. The method of claim 14 or 15, wherein the cancer is selected from pancreatic cancer, colorectal cancer, and lung cancer.

17. The method of any one of claims 14-16, wherein the cancer is drug-resistant or radiation-resistant.

18. The method of any one of claims 9-17, wherein said patient is pre-selected by utilizing an assay of said patient's tissue, blood or tumor for an abnormal, mutant or hyperactive ras gene or Ras protein, or an aberrant Ras-mediated biological process.

19. The method of any one of claims 9-18, wherein said patient's tissue, blood or tumor contains an abnormal, mutant or hyperactive ras gene or Ras protein, or aberrant Ras-mediated biological process.
